(19) United States
(12) Patent Application Publication
(10) Pub. No.: US 2025/0347698 A1
(43) Pub. Date: Nov. 13, 2025

(54) Multi-Omics Biomarker Detection System and Methods for Disease Diagnostics

(71) Applicant: Complete Omics Inc., Baltimore, MD (US)

(72) Inventors: Qing Wang, Baltimore, MD (US); Raghothama Chaerkady, Baltimore, MD (US); Liang Zhao, Baltimore, MD (US); Anirudh Kashyap, Baltimore, MD (US); Morgan Fair, Baltimore, MD (US)

(21) Appl. No.: 19/198,934

(22) Filed: May 5, 2025

Related U.S. Application Data
(60) Provisional application No. 63/643,536, filed on May 7, 2024.
(60) Provisional application No. 63/686,953, filed on Aug. 26, 2024.

(51) Int. Cl. G01N 33/68; G01N 33/92; G16B 40/10

(52) U.S. Cl. CPC G01N 33/6851; G01N 33/92; G16B 40/10; G01N 2570/00

(57) ABSTRACT

The present application provides methods and systems for single-run or minimally sequential detection and quantification of proteins, metabolites, and lipids using a unified instrumentation setup spanning a broad dynamic range (˜1 ng/L to 100 mg/L). In some embodiments, a two-phase database architecture transitions newly observed analytes from a discovery repository into a validated repository, enabling reproducible detection (CV<10%) with precise parameters (e.g., retention time, transitions). A machine-learning pipeline enhances automated peak selection by integrating large-scale manual curation with advanced feature extraction. The disclosed platform supports high-throughput multi-omics profiling of plasma or dried blood spot (DBS) samples and enables correlation with clinical factors such as age, BMI, or genetics. These systems maintain high sensitivity, scalability, and reproducibility, addressing long-standing limitations in clinical proteomics. As a result, the disclosed approach facilitates translational research, remote patient monitoring, and global healthcare implementation.

### CROSS REFERENCE TO RELATED APPLICATIONS

This application claims the benefit of U.S. Provisional Application Nos. 63/643,536 filed May 7, 2024 and 63/686,953 filed Aug. 26, 2024. The entire contents of the above-identified applications are hereby fully incorporated herein by reference.

This application also includes electronic submissions of the following tables, which are incorporated by reference in their entirety: Table 2. CompleteBank-Discovered Protein List.txt (117,997 bytes); Table 5. Reproducibility in 5 biological replicates.txt (1,391,391 bytes); Table 6. timsTOF HT analysis of three biological replicates.txt (553,170 bytes); Table 7. MyProt disease correlation.txt (942,180 bytes); Table 8. MyMeta-Polar Metabolite disease correlation updatedwithDBS.txt (196,726 bytes); Table 9. MyMeta-Lipid disease correlation updatedwithDBS.txt (309,492 bytes); Table 10. Selected Features and Corresponding p-Values for Each Disease Diagnosis.txt (98,270 bytes); Table 12. Complete360 with DBS samples.txt (1,294,942 bytes); Table 13. Metabolites and Lipids from DBS.txt (244,251 bytes); and Table 14. Clinical information of patients.txt (52,939 bytes). These tables are provided as tab-delimited text files in compliance with USPTO requirements and contain supporting data relevant to the examples and embodiments described herein.

LENGTHY TABLES
The patent application contains a lengthy table section. A copy of the table is available in electronic form from the USPTO web site (https://seqdata.uspto.gov/docdetail?docId=US20250347698A1). An electronic copy of the table will also be available from the USPTO upon request and payment of the fee set forth in 37 CFR 1.19(b)(3).

### TECHNICAL FIELD

The subject matter disclosed herein generally relates to multi-omics biomarker detection systems. More specifically, it pertains to integrated platforms utilizing mass spectrometry for the simultaneous detection of proteins, metabolites, and lipids to enhance disease diagnostics and biomarker validation.

### BACKGROUND

Biomarkers serve as measurable indicators of biological conditions and are widely used in disease diagnosis, treatment monitoring, and precision medicine. Blood-based biomarkers provide real-time snapshots of human health, making them highly valuable for clinical diagnostics. While nucleic acid-based biomarkers such as circulating tumor DNA have been widely adopted in oncology, many diseases, including cardiovascular, neurodegenerative, and autoimmune disorders, lack genetic markers. As a result, proteins and metabolites in blood serve as crucial disease indicators, offering a more dynamic and real-time reflection of physiological changes.

Traditional biomarker detection methods primarily focus on a single molecular class, such as proteins, metabolites, or nucleic acids, limiting their ability to provide a comprehensive molecular profile of a disease state. Existing mass spectrometry-based approaches suffer from low sensitivity, batch-to-batch variability, and limited scalability for clinical applications. Affinity-based detection methods, such as ELISA and immunoassays, can detect a limited subset of known proteins but lack the ability to perform unbiased, large-scale discovery. Meanwhile, untargeted mass spectrometry, despite its broad detection range, struggles with low reproducibility and limited sensitivity in complex biological samples.

One of the major limitations in current biomarker research is the challenge of integrating multi-omics data. Traditional approaches analyze proteins, metabolites, and lipids separately, requiring distinct workflows and specialized instrumentation. This compartmentalized analysis increases technical variability and reduces the ability to draw comprehensive conclusions from a single sample. The ability to quantify proteins, metabolites, and lipids in a single workflow is necessary to improve diagnostic accuracy.

Another limitation of current biomarker discovery and validation strategies is the lack of standardized, high-throughput approaches for iterative refinement of biomarker panels. Existing biomarker repositories are static and do not incorporate continuous validation across diverse clinical datasets. The platform in current application addresses this issue through its two-phase comprehensive database, which continuously refines biomarker parameters across more than 1,000,000 mass spectrometry runs to improve specificity and reproducibility.

There is a growing need for an integrated multi-omics biomarker detection system that overcomes these challenges by improving detection sensitivity, expanding biomarker coverage, and enabling reproducible validation across clinical and research settings. A system that integrates mass spectrometry-based proteomics, metabolomics, and lipidomics into a single workflow while leveraging a curated biomarker validation database would provide a significant advancement in biomarker-driven diagnostics and therapeutic monitoring.

This invention represents a transformative solution that integrates proteomic, metabolomic, and lipidomic analyses into a unified mass spectrometry-based workflow capable of detecting and quantifying over 10,000 human proteins and more than 2,000 small molecules across a broad dynamic range of physiological concentrations in body fluid samples (approximately 1 ng/L to 100 mg/L). By incorporating a discovery-to-validation biomarker database and a machine-learning model trained on hundreds of thousands of curated datasets, the system delivers reproducibility with coefficients of variation below 10%, while supporting automated disease classification with high diagnostic accuracy. This integrated approach addresses long-standing challenges in multi-omics assays, enabling real-time, scalable biomarker profiling for research and clinical applications.

Citation or identification of any document in this application is not an admission that such a document is available as prior art to the present invention.

### SUMMARY

The present disclosure provides a method for multi-omics biomarker detection in a single analytical pipeline. The method comprises obtaining a biological sample comprising proteins, metabolites, and lipids, wherein the sample is preserved for analyte detection across a concentration range from 1 ng/L to 100 mg/L. The sample is subjected to a unified preparation step that simultaneously removes high-abundance components and maintains the stability of proteins, metabolites, and lipids, wherein no separate instrumentation or reconfiguration is performed for individual biomolecular classes. Mass spectrometry-based detection of said proteins, metabolites, and lipids is performed in a single run or in multiple consecutive runs on the same instrumentation without major hardware reconfiguration. A machine-learning model, trained on at least hundreds of thousands of manually curated mass spectrometry datasets, automatically discriminates true analyte signals from noise, achieving a coefficient of variation of ten percent or less for repeated measurements. The resulting proteomic, metabolomic, and lipidomic data are compared to an iterative biomarker database that transitions biomarkers from a discovery stage to a validated stage upon meeting sensitivity and reproducibility thresholds. A disease-specific classification or biomarker panel is generated from the integrated multi-omics signals.

In some embodiments, processing the sample includes a two-step depletion protocol including chemical precipitation and antibody-conjugated resin depletion of high-abundance plasma proteins. In another embodiment, the biological sample is a dried blood spot, and said processing includes incubating said dried blood spot in a stabilization reagent at ambient temperature for at least three days without substantial biomarker degradation. In another embodiment, the mass spectrometry-based workflow operates across a dynamic range spanning about 1 ng/L to about 100 mg/L, enabling detection of ultra-low and high-abundance molecules in a single run.

In some embodiments, applying the data analysis pipeline includes training the machine-learning model on at least hundreds of thousands manually curated spectra, reducing the coefficient of variation below about 10%. In another embodiment, detection parameters are iteratively refined by repeating sample preparation, detection, and data analysis steps and updating the biomarker database upon meeting predefined reproducibility criteria. In another embodiment, identifying a disease-specific biomarker panel includes generating a receiver operating characteristic (ROC) curve with improved area under the curve (AUC) when integrating proteomic, metabolomic, and lipidomic features. In further embodiments, the method includes correlating one or more identified protein biomarkers with genetic variants via proteomic quantitative trait loci (pQTL) analysis, refining disease risk predictions.

In some embodiments, processing the sample further includes doping the sample with internal standard peptides for quantitative calibration of target analytes. In another embodiment, the mass spectrometry-based workflow employs dynamic multiple reaction monitoring (dMRM) that automatically adjusts collision energies in real time to enhance detection of low-abundance biomarkers.

The system disclosed herein supports the integrated implementation of this method. In some embodiments, the system comprises a unified sample preparation module configured to remove high-abundance components and preserve proteins, metabolites, and lipids from a single biological sample, wherein no separate instrumentation or reconfiguration is required for individual biomolecular classes. In another embodiment, the system includes a mass spectrometer assembly operable to detect proteins, metabolites, and lipids in one run or in multiple consecutive runs on the same instrumentation without major hardware reconfiguration across a concentration range from 1 ng/L to 100 mg/L, wherein said assembly detects said analytes without necessitating distinct hardware setups for proteomic versus small-molecule analysis.

In some embodiments, the system comprises a multi-phase biomarker database stored on at least one memory device, the database comprising discovery-phase entries and validated-phase entries. In another embodiment, a computing unit communicatively coupled to the mass spectrometer assembly and the biomarker database is programmed to: execute a machine-learning model trained on at least hundreds of thousands curated mass spectrometry datasets to distinguish analyte signals from noise with a quantification accuracy of coefficient of variation of ten percent or less; update said biomarker database by transitioning discovered biomarkers to validated-phase entries upon meeting predefined reproducibility thresholds; and generate a disease-specific classification or biomarker panel such that an area-under-the-curve (AUC) of at least 0.7 is achieved when distinguishing diseased samples from non-diseased samples.

In some embodiments, the sample preparation module comprises a chemical precipitation unit followed by an antibody-conjugated resin for selectively removing high-abundance plasma proteins. In another embodiment, the system further comprises a dried blood spot interface, wherein said sample preparation module includes a stabilization reagent adapted to minimize protein degradation for at least five days at ambient temperature. In another embodiment, the mass spectrometer assembly is configured to detect biomolecules over a dynamic range from about 1 ng/L to about 100 mg/L, enabling quantification of ultra-low abundance proteins.

In certain embodiments, the computing unit is programmed to execute a peak analysis model trained on over 1,000,000 mass spectrometry runs, achieving a reproducibility coefficient of variation below about 10%. In another embodiment, the biomarker database is iteratively updated based on repeated sample analyses, transitioning candidate biomarkers from a discovery phase to a validated phase upon meeting reproducibility thresholds. In yet another embodiment, the computing unit classifies disease states by selecting a subset of proteins, metabolites, and lipids that maximize diagnostic performance in a receiver operating characteristic (ROC) analysis, exceeding a preselected area under the curve (AUC) threshold.

In some embodiments, the system further comprises a pQTL analysis module integrated within the computing unit, configured to correlate identified protein biomarkers with genomic variants. In another embodiment, the mass spectrometer assembly is automatically tuned to adjust ionization parameters in real time through dynamic multiple reaction monitoring (dMRM), improving detection of low-abundance targets. In further embodiments, the computing unit applies internal standard peptides to ensure both relative and absolute quantification of proteins, metabolites, and lipids, enabling cross-run comparisons in a multi-omics dataset.

### BRIEF DESCRIPTION OF THE DRAWINGS

FIG. 1. The Complete360 platform enables high-throughput, blood-based proteomic and metabolomic profiling for diagnostic applications. FIG. 1A illustrates the Complete360 workflow, including blood sample processing, low-abundance protein enrichment, and extraction of proteins, metabolites, and lipids. Targeted disease panels are constructed using the CompleteBank database, followed by high-throughput mass spectrometry and automated data interpretation via the AI-powered CompletePeaking algorithm to generate diagnostic reports. FIG. 1B depicts the tissue origins of validated blood proteins, based on UniProt UP_TISSUE annotations, showing broad coverage across major human tissues and organs including brain, heart, liver, lung, kidney, pancreas, and immune cells. FIG. 1C presents subcellular localization and molecular class distribution: protein targets include cytoplasmic (34.5%), secreted (11.8%), cytoskeletal (9%), mitochondrial (8.2%), and other compartments, while small molecule targets include 762 polar metabolites from major biochemical classes and 1,395 lipid species spanning over 24 (sub) classes, such as TAG, DAG, FFA, PC, PE, and SM.

FIG. 2. Exceptional Sensitivity and Dynamic Range of Plasma Protein Detection Using the Complete360 Platform. FIG. 2 illustrates the wide dynamic range of plasma protein detection achieved by the Complete360 platform, spanning concentrations from ˜10 μg/mL to ˜100 μg/mL. This range encompasses nearly all known plasma proteins, overcoming one of the key analytical challenges in clinical proteomics. Detection was validated using well-characterized plasma proteins with known physiological concentrations. These results highlight the ability of Complete360 to achieve robust detection even with minimal sample input, enabling potential applications such as decentralized or in-home testing.

FIG. 3. Strong correlation between signal intensity, protein concentration, and measurement reproducibility in Complete360 assays. Quantitative analysis of over 4,500 proteins with known plasma concentrations reveals that reproducibility improves with increasing protein abundance. (Top) A positive relationship is observed between QqQ signal intensity and plasma concentration, reflecting the platform's quantitative sensitivity across a broad dynamic range. (Middle) Higher plasma concentration correlates with lower coefficient of variation (CV), demonstrating improved reproducibility at higher abundance. (Bottom) QqQ intensity also negatively correlates with CV (R²=0.51), indicating that signal strength is a reliable proxy for measurement consistency. These results validate the robustness of the Complete360® platform for reproducible protein quantification across eight orders of magnitude. Notably, the majority of proteins in the Complete360® panel lack documented blood concentration values, highlighting future directions for absolute quantification studies.

FIG. 4. Disease-Associated Molecular Signatures Identified by the Complete360® Platform. FIG. 4A presents principal component analysis (PCA) results of 102 plasma samples profiled by the Complete360 assay platform, covering patients with breast cancer, colorectal cancer, lung cancer, ovarian cancer, pancreatic cancer, prostate cancer, ulcerative colitis, and Alzheimer's disease. Distinct disease-specific clustering is observed, highlighting the platform's capacity to capture clinically relevant molecular variation. FIG. 4B displays ROC curve analyses generated using top-ranked features (based on t-test p-values) for each disease. Diagnostic models built with the top 1,000 multi-omics features-spanning proteins, polar metabolites, and lipids-consistently outperform protein-only models, as indicated by increased AUC values for most diseases. On average, the multi-omics panels consist of 794 proteins, 90 metabolites, and 117 lipids, demonstrating the advantage of integrating molecular layers to enhance diagnostic precision.

FIG. 5. Demographic and Physiological Influences on the Plasma Proteome Revealed by Complete360. Using the Complete360 platform, ultra-deep plasma proteomics was performed to evaluate associations between protein abundance and human demographic variables including age, sex, and body mass index (BMI). FIG. 5A-C present volcano plots showing the proteome-wide correlations with age, sex, and BMI, respectively. Numerous plasma proteins exhibit statistically significant shifts in abundance tied to these variables, reflecting physiological processes such as inflammation, extracellular matrix remodeling, lipid metabolism, and coagulation. In particular, the BMI-associated protein profile (FIG. 5C) includes TRIB3, INHBE, and ERBB4, all known to influence metabolic health, with leptin (LEP) emerging as a key driver. These findings underscore the sensitivity and predictive capacity of Complete360 for identifying molecular signatures relevant to personalized health monitoring, metabolic phenotyping, and early disease detection.

FIG. 6. Robust Proteomic Profiling of Dried Blood Spot (DBS) Samples Under Simulated Ambient Storage Conditions Using Complete360. FIG. 6A shows DBS sample cards collected from a single individual and stored in mailing envelopes at room temperature for 1 to 12 days to mimic real-world transport and storage conditions. Using the Complete360 platform, over 10,000 proteins were detected from these DBS samples. FIG. 6B compares the intensity profiles of protein biomarkers derived from DBS samples and matched plasma samples, demonstrating high similarity and minimal variation across storage durations. FIG. 6C presents the intensity profiles of metabolite and lipid biomarkers from the same matched plasma and DBS samples, further highlighting the consistency of molecular measurements across sample types and storage times. They both illustrate the similarity between proteomic profiles derived from DBS and matched plasma samples measured by Complete360, with minimal variation observed across storage durations. Approximately 47% of all proteins demonstrated a coefficient of variation (CV) below 25% across all DBS time points from the same individual, indicating high assay stability. Temporal expression analysis revealed that only 2-3% of proteins showed consistent directional changes, suggesting the majority of the proteome remains stable over ambient storage. These findings support the technical reproducibility and reliability of Complete360 for decentralized blood collection and proteomic analysis using DBS samples.

FIG. 7. Workflow for Dried Blood Spot (DBS) Sample Processing in the Complete360Platform. This schematic outlines the sample preparation protocol for DBS cards stored under ambient conditions and processed for proteomic analysis using the Complete360 platform. After DBS punches are incubated with TBS buffer containing 0.5% NP-40, the mixture is filtered to remove paper debris and centrifuged to collect soluble proteins. The extract is then treated with Minute Albumin Depletion reagent, and proteins are re-precipitated in 20% PEG-6000 to remove residual contaminants. Immunoglobulins and fibrinogen are sequentially depleted through Protein G affinity and heat-induced precipitation steps, respectively. The resulting protein solution undergoes S-Trap digestion to generate peptides for downstream LC-MS/MS analysis. This robust workflow enables high-yield and reproducible recovery of plasma proteins from DBS samples across various storage durations.

FIG. 8. Reproducibility analysis of Complete360 proteomic profiling across five biological replicates targeting 9,977 proteins. The median coefficient of variation (CV) for the entire panel is 11.97%. Among these, 7,833 proteins with CVs below 25% were consistently detected across all replicates, showing improved reproducibility with a median CV of 8.73%. A subset of 4,361 proteins exhibited high reproducibility with CVs below 10%, achieving a median CV of 4.77%. This robust performance highlights the potential of Complete360 for clinical translation upon validation of disease relevance.

FIG. 9. Discovery-mode analysis using the timsTOF HT platform identified 5,781 proteins from dried blood spot (DBS) samples, substantially expanding proteomic depth relative to conventional plasma-based analyses. Among these, 133 proteins were uniquely detected in DBS samples and were absent from both freshly frozen plasma and the existing CompleteBank database of 17,328 plasma proteins, highlighting the enhanced proteome coverage enabled by DBS profiling. 2,404 proteins are detected by timsTOF HT in both plasma and DBS samples, representing only about 30% of the total proteins. However Complete360 pipeline enables the detection of all 10598 proteins from both plasma and DBS samples demonstrating its ultra-high sensitivity and detectability across different sample types.

### DETAILED DESCRIPTION

### I. Overview

This disclosure relates to a multi-omics biomarker detection approach designed to simultaneously handle proteins, metabolites, and lipids in one analytical pipeline. Conventional methods often analyze these biomolecular classes in separate workflows or require major instrumentation reconfigurations between proteomics and metabolomics, leading to fragmented data, increased cost, and reduced reproducibility. By contrast, the present invention consolidates sample preparation, mass spectrometry detection, and data analysis into a single-run or minimally sequential setup, thereby covering a broad dynamic range of the physiological concentrations of disease biomarkers in body fluid samples (from about 1 ng/L to about 100 mg/L) within the same instrument arrangement.

A primary objective of the present invention is to address the longstanding challenges of insufficient sensitivity and poor reproducibility associated with conventional mass spectrometry-based proteomics and metabolomics, which have historically limited their clinical utility. This invention introduces a machine-learning-enabled data analysis pipeline, trained on a curated dataset comprising hundreds of thousands of mass spectrometry profiles, to achieve unprecedented analytical consistency and depth. The system targets a robust panel of over 10,000 proteins and more than 2,000+ metabolites—including approximately 1,300+ lipids and 700+ polar metabolites—each empirically validated for cross-sample detectability and reproducibility across diverse sample types and preparation protocols. The method consistently achieves a coefficient of variation below approximately 10%, establishing a new benchmark for precision. Additionally, the invention incorporates a dynamic biomarker validation framework, whereby candidate biomarkers are promoted to validated status upon satisfying stringent, predefined criteria for sensitivity and reproducibility. This iterative validation process enables the construction of clinically actionable, disease-specific panels that frequently achieve diagnostic classification performance with an area under the receiver operating characteristic curve (AUC) exceeding 0.85.

Another key goal of this invention is to overcome prior complexities arising from separate or partially overlapping omics pipelines. Therefore diagnostic panels of human disease utilizing multi-omics molecule, such as proteins, polar metabolites, and lipids can be detected and evaluated together.

In practice, the pipeline begins with a unified sample preparation protocol that preserves both low- and high-abundance analytes, preventing separate instrumentation for each biomolecular class. The mass spectrometry workflow can be conducted as one continuous run or multiple consecutive scans on the same hardware, avoiding major reconfiguration. Because proteins, metabolites, and lipids are simultaneously measured, the resulting multi-omics data feed into the machine-learning analysis, which reduces noise from co-elution and expands detection sensitivity across a wide concentration range. The final stage generates disease classifications or biomarker panels via a discovery-phase to validated-phase progression, facilitating large-scale clinical or research applications.

Throughout the following Detailed Description, the invention is described in terms of core components that include sample preparation, single-run or minimally sequential detection, multi-modal data analysis, iterative biomarker database refinement, and generation of disease-specific classification panels. The integrated system architecture includes a sample preparation module, a mass spectrometry-based detection system, a multi-phase biomarker database, and a computing unit equipped with machine-learning capabilities for signal discrimination and biomarker validation. Additional embodiments address specialized implementations such as dried blood spot sample handling, incorporation of internal standard peptides for calibration, and real-time instrument tuning to enhance detection sensitivity.

By streamlining multi-omics detection into a unified pipeline, this invention reduces operational complexity, improves reproducibility (CV<10%), and delivers clinically relevant biomarker panels for diverse diseases—a significant advancement over conventional single-omics or multi-instrument approaches.

### II. Multi-Omics Analysis

Multi-Omics Analysis refers to the integrated detection and interpretation of multiple molecular layers—proteins (proteomics), metabolites (metabolomics), and lipids (lipidomics)—in a single or minimally sequential analytical workflow. Conceptually, combining these distinct omics offers a holistic snapshot of an organism's physiological or pathological state, enabling more precise disease characterization and biomarker discovery than single-omics approaches alone. Numerous reviews—such as Hasin et al. (2017) (Genome Biol. 18:83), Karczewski & Snyder (2018) (Nat Rev Genet. 19:299-310), and Peng et al. (2021) ((Cell Rep. 37:109799)—underscore the theoretical power of multi-omics integration, showing how correlating data from diverse molecular classes can substantially enhance the detection of complex diseases (e.g., cancer, metabolic syndromes, neurological disorders).

However, existing multi-omics pipelines often rely on partial or post hoc data-level fusion. For instance, while Rampler et al. (2020) (Anal Chem. 12; 93(1):519-545) unify proteomics and some small-molecule metabolomics in one high-resolution MS approach, they typically exclude robust lipidomic coverage or require re-tuning of the instrument. Similarly, Resurreccion et al. (2022) (Metabolites. 27; 12(6):488) incorporate certain metabolites with proteomic analysis, yet omit a full tri-omics scope. Other studies, such as Zhang et al. (2020) (Proteomics 2020:e1900276), focus on computational multi-omics integration, merging separate LC-MS datasets rather than physically capturing proteomic, metabolomic, and lipidomic signals simultaneously. While these strategies confirm the conceptual advantage of combining data from different “-omics”, they generally do not address a unified or near single-run pipeline that physically detects proteins, metabolites, and lipids under one instrumentation arrangement with minimal or no hardware reconfiguration.

In contrast, the present invention delivers true tri-omics synergy (proteins, metabolites, lipids) in a single (or substantially single) mass spectrometry setup, bridging the gap between prior conceptual frameworks and an actual practical implementation. By spanning a dynamic range from about 1 ng/L to 100 mg/L in the same run, it captures both ultra-low abundance biomarkers (e.g., some plasma proteins, rare disease markers) and abundant lipid classes or metabolic intermediates with minimal scanning adjustments. Moreover, while prior references sometimes note the challenge of reliable “multi-omics” detection, they generally lack an iterative multi-phase biomarker database that transitions newly discovered biomarkers to validated-phase upon meeting reproducibility thresholds—an essential step for enabling clinical panels with an AUC often above 0.85.

Therefore, as defined herein, “Multi-Omics Analysis” extends beyond partial or purely computational data merges. Instead, it encompasses a one-instrument or minimal-run pipeline that physically detects and integrates signals from proteomics, metabolomics, and lipidomics at scale, empowered by a large, machine-learning-driven training dataset and iterative biomarker validation. This comprehensive, single-pipeline approach addresses the long-recognized limitations in prior art—improving reproducibility, reducing operational costs, and yielding more clinically actionable panels than the fragmented or post-hoc multi-omics methods described in existing literature.

### Sensitivity

Proteomics assays traditionally encounter difficulties detecting proteins that occur at low or ultra-low concentrations (e.g., sub-ng/mL) in biological fluids such as plasma. Multiple studies—including Aebersold & Mann (2003, Nature 422:198-207) and Domon & Aebersold (2006, Science 312:212-217)—have outlined the challenges of achieving deep coverage in complex samples, noting how high-abundance species often overshadow rare peptides and impede detection of minor proteins critical to clinical diagnostics. Although improvements in liquid chromatography (LC), mass spectrometry instrumentation, and sample preparation have incrementally raised detection thresholds, many conventional workflows still struggle to robustly quantify proteins below tens of ng/ml in plasma, unless extensive depletion or fractionation steps are employed. These fractionation-based methods can add complexity, reduce throughput, or risk losing analytes of interest.

In some embodiments, a multi-omics detection pipeline is employed to measure plasma proteins across a wide dynamic range—for instance, from approximately 10 ng/L to 100 mg/L—sufficient to span most physiologically relevant protein concentrations in clinical proteomics. This wide range addresses a longstanding challenge in plasma analyses, wherein proteins of vastly different abundances often co-exist, making it difficult to achieve the necessary depth and sensitivity. To evaluate the system's detection and reproducibility capabilities, a panel of 36 plasma proteins with well-characterized concentration profiles was analyzed across 12 biological replicates, encompassing a concentration range from 19 ng/L to 25 mg/L (Table 1). The data demonstrated that the present pipeline enables consistent quantitation across over six orders of magnitude in concentration.

The platform achieved high reproducibility, with an average coefficient of variation (CV) of 3.92%, ranging from 1.4% to 7.0% across all proteins analyzed (Table 1). In certain embodiments, this level of reproducibility is illustrated by representative raw spectra of key protein biomarkers. For example, Isocitrate dehydrogenase [NAD] subunit beta (Uniprot ID: 043837), normally annotated around 8.3 ng/L in plasma, was consistently detected even under 1:1,000 dilution conditions, highlighting the platform's capacity for detecting low-abundance targets. In some embodiments, the lowest protein concentration reliably detected in the dataset was approximately 3.5 ng/L, further reinforcing the pipeline's sensitivity for ultra-low abundance analytes.

TABLE 1

| | | | | CV | |
|---|---|---|---|---|---|
| | | | | observed | |
| | | | | from | Average |
| | | | Gene | Complete | Concentration |
| Sequence | Uniprot ID | Protein Name | Name | 360 | (ng/L) |
| 1 | Q86X55 | Histone-arginine | CARM1 | 5.9% | 19 |
| | | methyltransferase CARM1 | | | |
| 2 | Q8N1Q1 | Carbonic anhydrase 13 | CA13 | 4.0% | 27 |
| 3 | Q17RW2 | Collagen alpha-1(XXIV) chain | COL24A1 | 5.2% | 52 |
| 4 | P46459 | Vesicle-fusing ATPase | NSF | 6.0% | 59 |
| 5 | Q9Y2Q3 | Glutathione S-transferase kappa 1 | GSTK1 | 2.0% | 110 |
| 6 | P58499 | Protein FAM3B | FAM3B | 5.6% | 150 |
| 7 | Q9C0C9 | (E3-independent) E2 ubiquitin- | UBE20 | 4.2% | 220 |
| | | conjugating enzyme | | | |
| 8 | O60493 | Sorting nexin-3 | SNX3 | 5.7% | 270 |
| 9 | O43915 | Vascular endothelial growth factor | VEGFD | 1.4% | 410 |
| | | D (VEGF-D) | | | |
| 10 | Q16531 | DNA damage-binding protein 1 | DDB1 | 2.2% | 440 |
| 11 | P22223 | Cadherin-3 | CDH3 | 5.8% | 510 |
| 12 | Q15185 | Prostaglandin E synthase 3 | PTGES3 | 3.4% | 660 |
| 13 | P51858 | Hepatoma-derived growth factor | HDGF | 1.9% | 790 |
| 14 | Q9H3T3 | Semaphorin-6B | SEMA6B | 3.2% | 1100 |
| 15 | Q9Y613 | FH1/FH2 domain-containing | FHOD1 | 2.4% | 1300 |
| | | protein 1 | | | |
| 16 | P19338 | Nucleolin | NCL | 2.9% | 1400 |
| 17 | P30153 | Serine/threonine-protein | PPP2R1A | 3.3% | 2000 |
| | | phosphatase 2A | | | |
| 18 | Q8IUX7 | Adipocyte enhancer-binding | AEBP1 | 4.4% | 2700 |
| | | protein 1 | | | |
| 19 | Q9UHG2 | ProSAAS | PCSK1N | 2.3% | 3300 |
| 20 | Q96C24 | Synaptotagmin-like protein 4 | SYTL4 | 2.3% | 3300 |
| 21 | Q6NUS6 | Tectonic-3 | TCTN3 | 5.2% | 4400 |
| 22 | P21399 | Cytoplasmic aconitate hydratase | ACO1 | 2.3% | 10000 |
| | | (Aconitase) | | | |
| 23 | Q15691 | Microtubule-associated protein | MAPRE1 | 4.6% | 11000 |
| | | RP/EB family member 1 | | | |
| 24 | P08319 | All-trans-retinol dehydrogenase | ADH4 | 2.3% | 20000 |
| | | [NAD(+)]ADH4 | | | |
| 25 | O15204 | ADAM DEC1 | ADAMDEC1 | 2.2% | 28000 |
| 26 | P14923 | Junction plakoglobin | JUP | 2.2% | 64000 |
| 27 | P02008 | Hemoglobin subunit zeta | HBZ | 5.9% | 80000 |
| 28 | P04211 | Immunoglobulin lambda variable | IGLV7-43 | 5.5% | 130000 |
| | | 7-43 | | | |
| 29 | P06753 | Tropomyosin alpha-3 chain | TPM3 | 7.0% | 170000 |
| 30 | P55056 | Apolipoprotein C-IV | APOCA | 3.0% | 1000000 |
| 31 | P01591 | Immunoglobulin J chain | JCHAIN | 4.2% | 2200000 |
| 32 | Q6UXB8 | Peptidase inhibitor 16 | PI16 | 4.1% | 2900000 |
| 33 | A0A087WSY6 | Immunoglobulin kappa variable | IGKV3D- | 6.5% | 5800000 |
| | | 3D-15 | 15 | | |
| 34 | P60709 | Actin, cytoplasmic 1 | ACTB | 3.8% | 5800000 |
| 35 | P01602 | Immunoglobulin kappa variable 1- | IGKV1-5 | 4.1% | 8400000 |
| | | 5 | | | |
| 36 | P02750 | Leucin-rich alpha-2-glycoprotein | LRG1 | 2.3% | 25000000 |

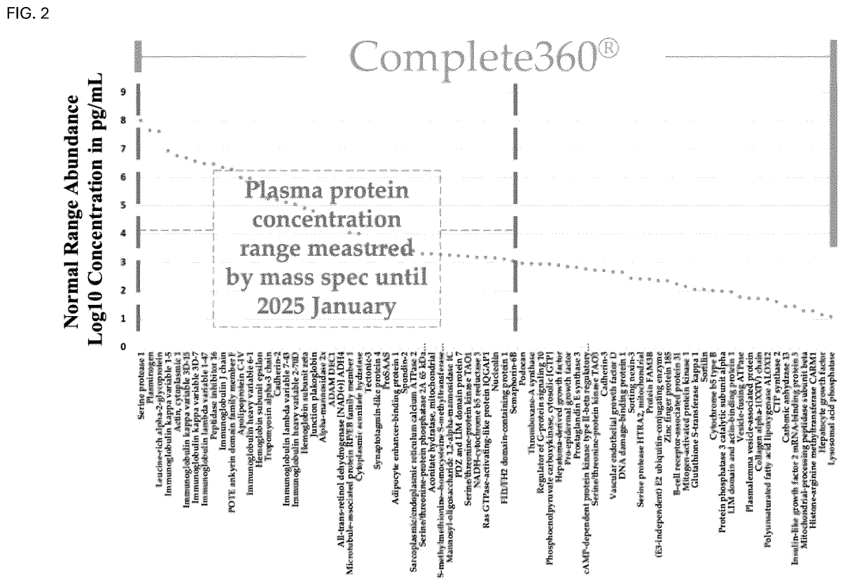

In further embodiments, FIG. 2 highlight this extensive range and demonstrate how selected low-abundance proteins produce strong, reproducible signals under optimized conditions. For example, proteins such as Leukocyte cell-derived chemotaxin 1 (Uniprot ID: 075829), NADH dehydrogenase [ubiquinone] flavoprotein 2 (Uniprot ID: P19404), Calretinin (Uniprot ID: P22676), and Methionine aminopeptidase 1 (Uniprot ID: P53582) similarly displayed high-quality peak profiles below ˜10 ng/L. Observations of robust signal intensities for these proteins at such low concentrations underscore the pipeline's capacity to detect analytes otherwise inaccessible in conventional plasma proteomics.

In some embodiments, the ability to capture these ultra-low abundance species is particularly beneficial for novel applications requiring reduced sample inputs or simplified collection methods (e.g., in-home or remote sampling). While prior approaches have noted difficulties establishing a consistent “baseline” plasma concentration for certain proteins due to methodological variations, the present system offers a more standardized and reproducible framework, aiming to mitigate discrepancies. As the pipeline continues refining detection parameters, additional calibration or reference panels may be introduced to define improved normal baseline values for plasma proteins at low concentrations.

Overall, these findings confirm that, in some embodiments, the disclosed pipeline achieves excellent detection performance over a broad dynamic range and sub-ng/L threshold sensitivities for multiple proteins in plasma. Beyond enumerating these capabilities, such a robust detection framework permits expansion to other analyte classes, including lipids and metabolites, maintaining similarly high sensitivity and reproducibility.

### Reproducibility

In clinical proteomics, reproducibility remains paramount, as variability in detection can undermine diagnostic validity. Numerous studies highlight the difficulty of achieving consistent results at large scale. For example, Aebersold & Mann (2016, Nature 537:347-355) discuss how multi-run variability hampers the broader translational use of mass spectrometry data, while Gillet et al. (2012, Mol. Cell. Proteomics 11:0111.016717) note that even data-independent acquisition approaches can face reproducibility gaps if not accompanied by rigorous normalization and iterative parameter refinements. The widespread use of extensive signal-amplification or fractionation steps in some pipelines can introduce further sources of error, complicating the standardization needed for clinically oriented assays.

In some embodiments, the disclosed multi-omics pipeline achieves high reproducibility, a critical factor for clinical diagnostics. Although certain proteomics methods may attain substantial sensitivity, their adoption in clinical settings can be hindered by insufficient assay consistency-particularly where extensive signal amplification techniques (e.g., NGS-based readouts) introduce additional variability. Here, the focus is on confirming reproducibility across a broad range of protein concentrations in plasma.

In some embodiments, the present invention addresses these reproducibility constraints by systematically evaluating its multi-omics detection pipeline across varied protein abundance ranges—from about 19 ng/L to about 25 mg/L—and across multiple biological replicates. For instance, one illustrative approach tests a panel of 36 proteins with documented plasma concentrations across 12 biological replicates, demonstrating an average coefficient of variation (CV) below approximately 4%, with a range from about 1.4% to 7%. These values significantly surpass typical literature benchmarks for large-scale plasma proteomic runs, wherein CVs frequently exceed 10-15% (see, e.g., Jiang et al., ACS Meas Sci Au. 2024 Jun. 4; 4(4):338-417). The system's reliability is further exemplified by raw spectral data, enabling consistent detection of both high-abundance (mg/L level) and low-abundance (ng/L level) targets in the same or minimally sequential runs.

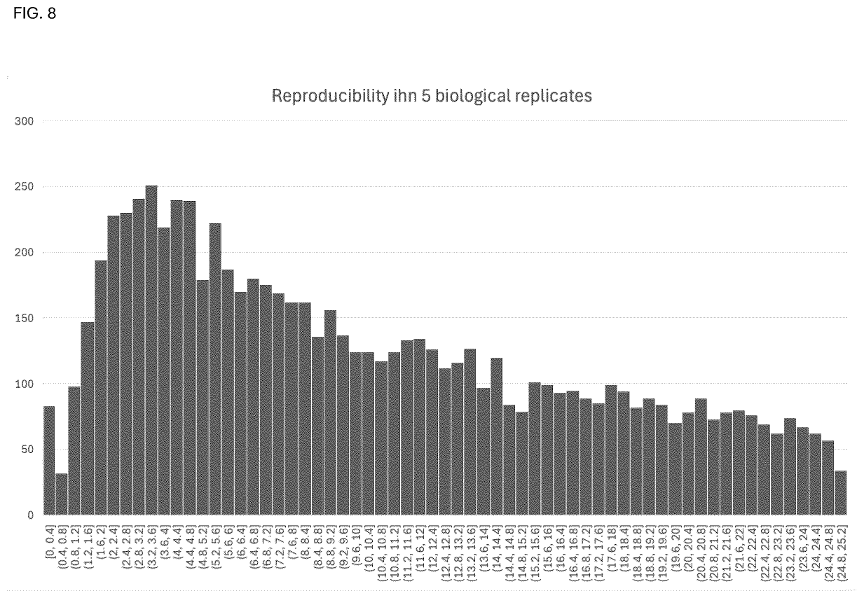

To confirm that these results extended beyond a small panel, further embodiments targeted 9,977 proteins across five biological replicates (see, e.g., FIG. 8 and Table 5). The median CV for the entire panel was approximately 12%, with a substantial fraction (about 7,833 proteins) showing CVs below 25%, and 4,361 proteins (median CV ˜4.8%) under 10%. Such results highlight the platform's capacity for large-scale reproducibility, positioning these assays as a viable basis for proteomics-based clinical diagnostics once suitable disease relevance is established.

Collectively, these findings illustrate that, in some embodiments, the method or system herein disclosed combines wide dynamic range coverage with consistent, sub-10% CV reproducibility, thus addressing key obstacles in clinical proteomics applications. The reliability achieved on both small, carefully selected panels and broader protein sets underscores the adaptability of this approach. Consequently, in some embodiments, this pipeline enables robust translational research, guiding future diagnostic or therapeutic monitoring applications grounded in precise, reproducible protein quantification from plasma samples.

### Multi-Omics Integration for Enhanced Diagnostic Precision

In certain embodiments, a single-run or minimally sequential pipeline is employed to measure proteins, metabolites, and lipids from the same biological sample, thereby forming a multi-omics dataset that substantially elevates diagnostic accuracy. Literature recognizes the need for multi-omics integration—Karczewski & Snyder (2018, Nat. Rev. Genet. 19:299-310) note that analyzing disparate “omics” can uncover complex disease mechanisms, yet most methods either focus on specific pairs (e.g., proteomics+targeted metabolomics) or rely on post-hoc data-level fusion. For example, Rampler et al. (2020) (Anal Chem. 12; 93(1):519-545) describe combining proteomic and small-molecule detection in a single high-resolution platform but do not incorporate broad lipid coverage, while other studies rely on separate instrumentation or reconfiguration to measure different molecular classes.

In contrast, the present invention integrates metabolomic and lipidomic analyses concurrently with proteomics in one instrumentation workflow, leveraging an optimized mass spectrometry assay for each class. In some embodiments, approximately 762 metabolites and 1,395 lipids are detected—covering both polar metabolites and lipid species across multiple subclasses. Data from these assays may undergo distinct normalization schemes (e.g., median normalization for metabolites vs. separate procedures for lipids), yet the measurements themselves remain part of the same overarching pipeline. By consolidating analyses onto a unified platform, this approach supports easier clinical adoption and reduces sample usage compared to conventional multi-platform setups. For instance, polar metabolites and lipid species may undergo separate normalization procedures, as set forth in FIG. 8, Table 5 and Table 6, respectively. This multi-omics synergy allows for correlative analyses between metabolic and protein signals, thus yielding disease-specific insights anchored by biologically relevant pathways. In one illustrative example, the pipeline reveals strong agreement (average 69% overlap) in the top 10 metabolomic pathways identified from both proteomic and metabolomic perspectives, signifying the robust multi-omics coverage.

To harness the pipeline's capacity fully, some embodiments incorporate peptides, metabolites, and lipids as collective features for disease classification. A t-test may be performed to compare each condition against all other cohorts, generating ranked p-values and systematically selecting the top 1,000 features for model construction. In certain implementations, separate receiver operating characteristic (ROC) curves compare classification outcomes when only proteomic (peptide) features are used versus when all three molecular layers are integrated. These analyses reveal increased area under the curve (AUC) values upon including both metabolomic and lipidomic features, thereby highlighting the advantage of combining multiple molecular layers for disease detection.

Overall, the simultaneous multi-omics approach described herein provides enhanced diagnostic precision, leveraging cross-corroboration among proteins, metabolites, and lipids in one unified workflow. This contrasts with conventional single-omics or narrowly combined approaches, which often overlook synergistic relationships among different biomolecular categories. By allowing broad and flexible integration of metabolite and lipid measurements into a proteomic pipeline, this system significantly elevates the predictive strength (represented by a higher AUC of the ROC) of disease models and fosters more meaningful clinical and research applications.

### III. Biomarker

Biomarker in the present context encompasses any quantifiable molecule—be it a protein, small-molecule metabolite, or lipid—that reliably indicates a physiological or pathological condition. Historically, biomarker research focused on single classes of molecules, often identified through targeted immunoassays or narrow-range mass spectrometry. For instance, Tirumalai et al. (2003) (Mol. Cell Proteomics 2(10):1096-1103) provided an early overview of low molecular weight proteins in serum as potential biomarkers for cancer, emphasizing how certain proteins appear at ng/ml (or even lower) concentrations yet exhibit critical diagnostic significance. While these pioneering efforts highlighted the clinical utility of protein biomarkers, they generally bypassed concurrent assessment of lipid or metabolite levels, thus limiting insight into broader disease pathways.

Over the past two decades, researchers have begun recognizing that multi-dimensional biomarker sets often yield richer information than single-target assays. For example, Wang et al. (2019) (Bioinformatics 35(14): 2644-2652) describe how machine-learning integration of metabolite data can boost disease classification accuracy, but do so by merging metabolomics signals in an exclusively separate data pipeline from proteomic analysis. This partial integration underscores a persistent challenge: even advanced computational workflows typically rely on distinct instrumentation setups for different molecular classes, making truly unified biomarker discovery an operational hurdle.

Meanwhile, large-scale initiatives such as the Human Proteome Project have mapped enormous numbers of potential protein biomarkers, and parallel efforts in metabolomics (e.g., the HMDB) and lipidomics (e.g., Lipid Maps) have similarly documented thousands of candidate disease markers. Despite these efforts, very few studies systematically capture all relevant molecular types (proteins, metabolites, lipids) within a single or minimally sequential analytical run. Such an approach is vital for uncovering correlated biomarker panels, where, for instance, an inflammatory protein might appear in tandem with a specific lipid class and a metabolic byproduct, collectively defining a more predictive signature than any single molecule alone.

In addition to coverage across multiple “omis,” reproducibility in biomarker quantification is crucial for clinical translation. Studies like Karczewski & Snyder (2018) (Nat. Rev. Genet. 19:299-310) illustrate that even well-studied biomarkers can fall short of clinical standards without stringent validation. Traditional workflows, though they may find protein markers or metabolic indicators, tend to lack a built-in iterative validation pipeline capable of progressively transitioning newly discovered analytes from “candidate” to “clinically validated.” This gap can prolong or derail the movement of promising biomarkers into real-world usage.

Moreover, post hoc or partial data integration frequently ignores dynamic interplay among molecular species that might emerge when measured simultaneously. A newly found protein, for example, might seem insignificant until correlated with a lipid sub-class and a disease-specific metabolite spike—an interplay that remains hidden if separate instrumentation or re-tuning is required for each analyte class. While integrative approaches exist at the bioinformatics level, they typically patch together data gathered under varying conditions and scanning parameters, risking missing subtle yet pivotal co-fluctuations of molecules across proteomic, metabolomic, and lipidomic layers.

The present pipeline interprets any clinically pertinent analyte—including but not limited to inflammatory proteins (e.g., C-reactive protein, interleukins), cardiac markers (troponin, BNP), hormones (insulin, cortisol), small-molecule metabolites (lactate, amino acids like phenylalanine or tryptophan, organic acids such as 2-hydroxyglutarate), and lipids (cholesterol, sphingolipids, ceramides)—as potential biomarkers, so long as they exhibit diagnostic or prognostic significance for a given disease context. For example, a panel combining certain high-sensitivity proteins (like CRP or troponin at ng/L levels), a subset of lipid species (e.g., ceramides implicated in cardiovascular risk at μg/mL range), and key metabolites (like lactate or branched-chain amino acids) can together form a multi-omics signature far more informative than any one marker in isolation. Traditional workflows, however, often measure each category separately or in partially integrated approaches, risking missed correlations that could distinguish subtle disease phenotypes.

By harnessing a single-run or minimal-run workflow, the invention's pipeline quantifies these diverse biomarker classes under uniform instrument conditions and data analysis steps. A high-abundance lipid such as total triglycerides in the mg/mL range is measured simultaneously with sub-ng/mL proteins known to indicate acute inflammatory responses (e.g., certain interleukins), thus allowing real-time correlation of molecules typically handled via separate assays. Moreover, while conventional single-omics studies can identify biomarkers like C-reactive protein alone, they might overlook how its elevation coincides with a particular ceramide ratio and a distinct metabolic fingerprint—an interplay potentially crucial for, say, advanced cardiovascular risk or inflammatory states. The invention's approach addresses that gap by enabling a large-scale, machine-learning-assisted curation that systematically evaluates each analyte's reproducibility (coefficient of variation below ˜10%) and diagnostic performance (AUC≥0.85), thereby consolidating newly uncovered multi-omics biomarkers into an iterative discovery-to-validation cycle.

Thus, “biomarker” here is not just a single-protein measure or a sole metabolic ratio, but can include practically any relevant protein, lipid, or metabolite quantifiable within the ˜1 ng/L to 100 mg/L dynamic range—validated in the same instrument pass or minor consecutive passes, eliminating the typical fragmentation seen in separate proteomic vs. lipidomic vs. metabolomic workflows.

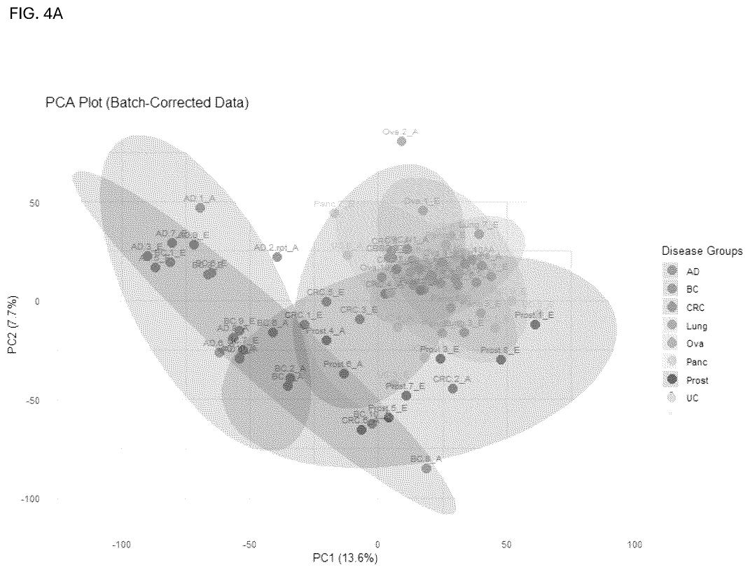

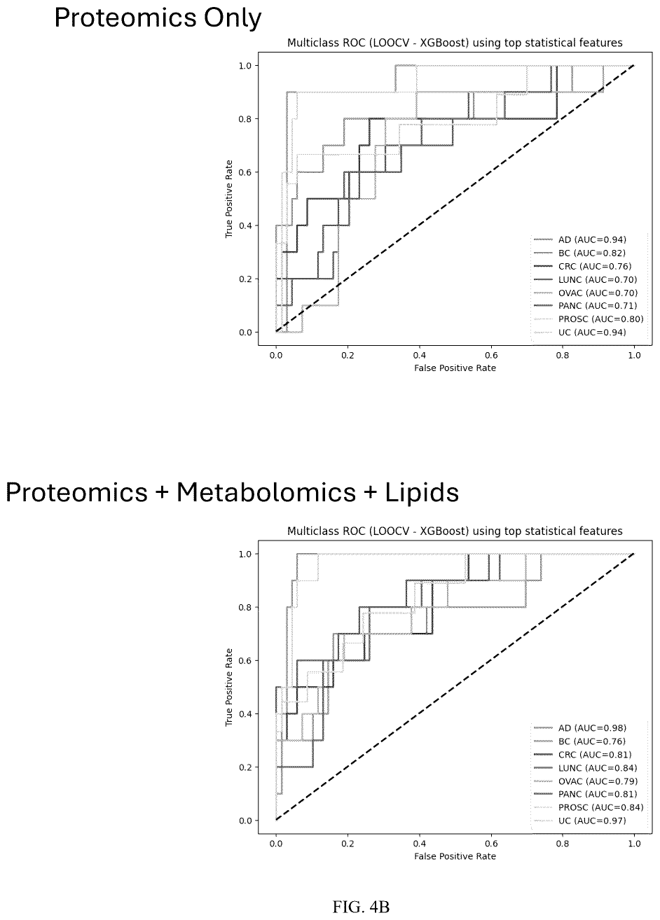

In certain embodiments, an integrated multi-omics pipeline, as disclosed herein, was applied to ninety plasma samples obtained from biobanks, each sample corresponding to patients diagnosed with one of eight distinct diseases: breast cancer, colorectal cancer, lung cancer, ovarian cancer, pancreatic cancer, prostate cancer, ulcerative colitis, or Alzheimer's disease. Upon data acquisition and subsequent machine-learning-based analysis, the pipeline showed clear disease clustering within their respective categories, as illustrated in FIG. 4. Further statistical and computational investigations revealed multiple biomarker patterns unique to each disease.

For example, the pipeline identified 386 potential biomarkers specific to breast cancer, 288 for colorectal cancer, 226 for lung cancer, 209 for ovarian cancer, 195 for pancreatic cancer, 331 for prostate cancer, 280 for ulcerative colitis, and 407 for Alzheimer's disease, as summarized in Table 7. A substantial fraction of these markers appeared consistent with previous literature, suggesting feasibility and alignment with established disease-related molecules. Specifically, 319, 199, 172, 121, 112, 192, 64, and 246 of the biomarkers detected across these eight diseases had been documented elsewhere, indicating that the invention's pipeline can effectively confirm known biomarkers under a single-run or minimal-run approach.

In certain embodiments, the pipeline's multi-omics coverage, dynamic range of approximately 1 ng/L to 100 mg/L, and iterative biomarker validation collectively enable the discovery of biomarkers not yet described in the context of each disease. For instance, the analysis found 17%, 31%, 24%, 42%, 43%, 42%, 77%, and 40% of the biomarkers identified for breast, colorectal, lung, ovarian, pancreatic, prostate, ulcerative colitis, and Alzheimer's disease, respectively, were previously unreported. By capturing proteins, metabolites, and lipids concurrently and applying a large-scale machine-learning curation with reproducibility thresholds (coefficient of variation ≤10%), the pipeline disclosed herein significantly expands the biomarker repertoire relative to partial or single-omics methods.

Moreover, individual embodiments illustrated disease-specific findings. In breast cancer, key biomarkers linked to progression and metastasis included eIF4E type 2, vinculin, Serpin B6, and Ficolin-2. Literature references indicate that eIF4E type 2 overexpression frequently correlates with aggressive breast carcinoma phenotypes, while vinculin is involved in cell migration and metastasis (see, for example, references [25, 26]). Further, Serpin B6 is associated with protease regulation impacting metastasis [27], and Ficolin-2, an immune-system component, confers enhanced immune surveillance. In another embodiment, colorectal cancer panels included biomarkers such as Alanine aminotransferase 2 (ALT2) and C-C motif chemokine 19 (MIP-3-beta), documented in prior works [31, 32] to be implicated in tumor progression or hepatic metastases, along with newly discovered or lesser-known markers.

The pipeline likewise demonstrated synergy for lung cancer, ovarian cancer, pancreatic cancer, prostate cancer, ulcerative colitis, and Alzheimer's disease, each exhibiting distinctive biomarker patterns—some previously established in the literature, and others newly revealed by the integrated tri-omics approach. For instance, in Alzheimer's disease, proteins such as LRRTM4 and NADH dehydrogenase [ubiquinone] 1 alpha subcomplex subunit 5, correlated with synaptic function and mitochondrial dysfunction, respectively, emerged alongside novel metabolite-lipid correlations not identifiable by single-layer detection. As a result, each disease's biomarker panel can be refined through iterative database validation for reproducibility (CV<10%) and diagnostic performance (AUC≥0.85).

Thus, in contrast to references that typically limit detection to proteomics or partial metabolomics, the invention's pipeline captures a broader and more comprehensive set of disease markers simultaneously. This synergy is particularly beneficial for diseases with complex etiologies where proteomic, metabolomic, and lipidomic signals jointly define disease progression. By enabling robust cross-omics correlation within one instrumentation setup or minimal sequential runs, the invention advances clinical and research capabilities to identify, confirm, and expand biomarker panels across a wide range of disease states.

### IV. Optimized Target Detection Strategies Based on DDA and DIA Profiling for Enhanced Sensitivity and Reproducibility

Large-scale protein databases have long been fundamental to proteomics research. Early initiatives, such as the PeptideAtlas (Desiere et al., 2006, Nucleic Acids Research 34:D655-D658) and the Human Proteome Organization (HUPO) projects, aimed to map out all human proteins using standardized LC-MS methods. Subsequent releases of public repositories, including PRIDE (Vizcaíno et al., 2016, Nucleic Acids Research 44:D447-D456), popularized data sharing and partial validation, but often lacked continuous or iterative refinement for each identified protein. Similarly, the Human Proteome Project (Omenn et al., 2014, Journal of Proteome Research 13:661-667) consolidated extensive proteomic identifications with annotation layers, yet each library update typically presented static detection parameters that might not adapt to newly optimized methods or subtle improvements in instrument performance. In parallel, curated reference spectra libraries (e.g., GPMDB, MassIVE) emerged for targeted or spectral library-based searches, though these references usually reflect single-phase acquisitions—meaning they provide detection data but not continuous re-validation with fresh instrumentation runs.

Professional practice in proteomics has therefore recognized the need for robust, iteratively updated repositories that refine detection conditions (retention times, collision energies, transitions) alongside comprehensive metadata about sample preparation or instrumentation changes. For example, Gillet et al. (2012, Molecular & Cellular Proteomics 11:O111.016717) introduced data-independent acquisition strategies that advocated updating detection libraries whenever new runs were performed, albeit not consistently integrated as a multi-phase “discovery-plus-validation” pipeline. Even more advanced proteomic-lipidomic cross-references (Rampler et al. 2020. Anal Chem. 12; 93(1):519-545) remain relatively static in how they store parameters, seldom re-optimizing detection thresholds across thousands of additional runs. Consequently, while the concept of building big proteomic databases is well established, a multi-round iteration explicitly verifying or re-validating each potential analyte in multi-omics contexts has remained sparse.

Various mass spectrometric acquisition strategies exist to detect and quantify proteins, metabolites, and lipids in complex samples. Historically, data-dependent acquisition (DDA)—has been widely used in proteomic research. In DDS-MS, the instrument automatically selects and fragments the most abundant precursor ions within each survey scan, creating product-ion spectra for a limited subset of the molecules present. While this targeted fragmentation approach can yield high-quality spectra for prevalent analytes, it often misses lower-abundance ions that fail to rank among the top intensities in each cycle. Consequently, while DDS-MS has contributed significantly to proteomics, it may yield less comprehensive coverage in samples containing a broad dynamic range (e.g., 1 ng/L to 100 mg/L), especially if high-abundance proteins and lipids overshadow subtle analytes.

In recent years, data-independent acquisition (DIA) methods—sometimes referred to as SWATH-MS, MSE, or DIA-PASEF—have emerged as an alternative. DIA simultaneously fragments all precursor ions in defined m/z or ion mobility windows without exclusively focusing on top-intensity ions, potentially capturing a more complete molecular profile in a single run. Nevertheless, DIA approaches also bring challenges, such as complex mixed-fragment spectra that demand advanced deconvolution algorithms or large spectral libraries for correct identification. Meanwhile, some hybrid systems rely on data-dependent triggers for only a portion of the scan cycle, or combine targeted MRM transitions with data-independent scans to achieve partial coverage of known biomarkers.

DDA and DIA are widely utilized mass spectrometric strategies for proteomic profiling. While DDA offers high-quality spectra for selected precursor ions, it may overlook low-abundance analytes. In contrast, DIA enables broader coverage by concurrently fragmenting all ions within specified mass ranges, albeit with increased complexity in spectral deconvolution. Both approaches have been foundational to proteomics, yet each carries inherent limitations when applied in isolation.

To achieve the highest sensitivity and reproducibility in complex plasma proteomics, the present invention introduces an optimized target detection approach that integrates features of DDA and DIA profiling. This strategy combines the depth of DDA-based identification with the breadth of DIA-based acquisition to enhance coverage across a wide dynamic range. By refining detection conditions—such as retention time windows, collision energies, and scan scheduling—and continuously updating spectral libraries, the method enables robust quantification of both high- and ultra-low-abundance proteins with superior consistency.

Against this backdrop, certain embodiments of the current invention integrate the strengths of both data-dependent and data-independent scanning to maximize coverage of proteins, metabolites, and lipids in a single or minimally sequential run. In some embodiments, the pipeline uses DDS-MS for in-depth characterization of high-interest ions identified in an earlier library search, while complementary strategies akin to DIA or carefully scheduled selected reaction monitoring (SRM/dSRM) ensure consistent coverage across a wide dynamic range. By leveraging multiple scanning modes in a coordinated fashion and employing an iterative, large-scale database curation, the invention can minimize data gaps commonly observed under exclusively data-dependent or data-independent approaches.

Thus, while DDA-MS has been crucial for proteomic identification and data-rich MS/MS spectra, it can fall short in samples with vast dynamic range or unknown molecular complexity—particularly in multi-omics scenarios capturing lipids and metabolites beyond the typical scope of DDA-based proteomics. By contrast, the present invention's iterative pipeline broadens the coverage to incorporate low-abundance biomarkers often ignored in purely data-dependent scans, employing carefully refined transitions and reproducible detection parameters validated through thousands (or hundreds of thousands) of mass spectrometry runs.

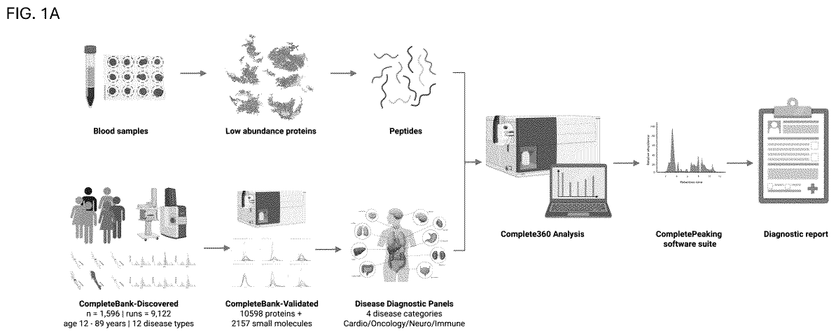

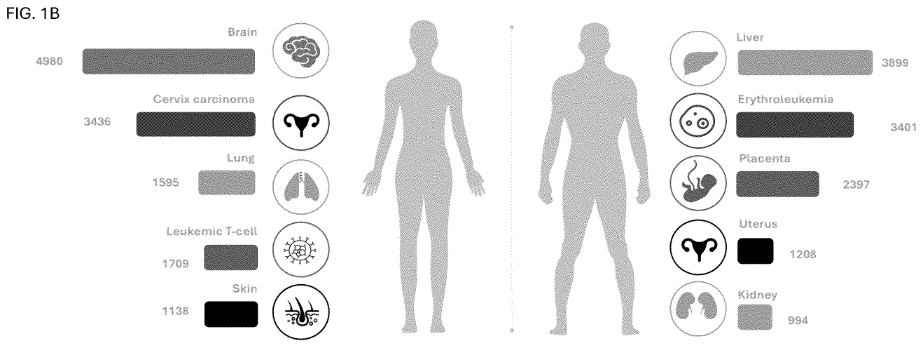

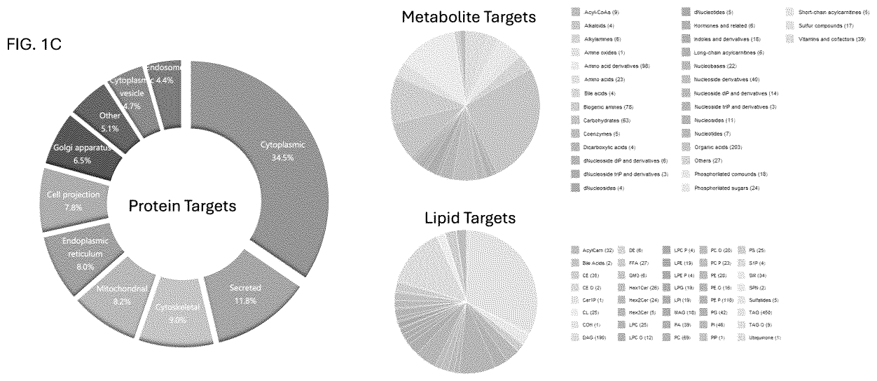

In some embodiments, a two-phase database methodology is employed to enable systematic detection and validation of proteins and small molecules in human plasma. In the first, or discovery, phase, a repository of about 16,743 proteins and 2,927 metabolites or lipids is assembled from extensive plasma analyses, thereby establishing preliminary detection parameters-such as retention times, mass-to-charge values, and putative transitions—for each candidate analyte. This collection is hereinafter referred to as a “discovery module,” which, in certain embodiments, is exemplified in FIG. 1A. By comparing the discovery module's protein list with external resources (e.g., the Human Proteome Project (HPP) database), numerous proteins, including ˜536 from the PE2-5 groups, may be newly identified in plasma, emphasizing the breadth and completeness of this foundational dataset.

The second phase, sometimes called the validation phase, provides rigorous confirmation of each discovery-phase analyte's reliability and reproducibility. In certain embodiments, each protein or metabolite undergoes at least five iterative rounds of optimization, targeting improvements in detection sensitivity (e.g., signal-to-noise ratio), retention time alignment, and collision energy tuning. Data from each round are meticulously curated, removing potential artifacts and refining final transitions. Through this multi-step refinement, a validated repository emerges, comprising, for example, 10,598 plasma proteins and 2,208 small molecules. This validated repository includes precise detection settings for each analyte, covering recommended retention times, m/z values for MS1 and MS2 fragmentation, and optimized collision energies. More than 9,000 time-of-flight (TOF) LC-MS/MS runs and over 1,000,000 triple quadrupole (QqQ) MS runs may be employed during these phases, supplemented by manual review of upward of 600,000 QqQ raw spectral files to ensure consistent performance and minimal coefficients of variation.

In certain embodiments, tissue annotation analyses are performed on validated proteins to confirm their diverse origins across major human tissues or organs. Subcellular localization studies can further reveal a distribution spanning cytoplasmic, secreted, mitochondrial, endoplasmic reticulum, and additional compartments. For instance, FIG. 1B may illustrate the fraction of validated proteins from each subcellular category, while FIG. 1C can show the distribution of ˜2,157 small molecules encompassing 762 polar metabolites and 1,395 lipid species, each subgroup covering various metabolic, drug-related, or disease pathways.

During method development, spanning approximately six years and documenting in excess of 9,000 body fluid samples, a manual curation approach is employed to fine-tune detection parameters and minimize measurement variability. In some embodiments, this manual curation is used to train an AI-based learning and peak-picking system (also shown conceptually in FIG. 1A), wherein comprehensive pattern-recognition algorithms identify precise peaks for each target while addressing co-eluting molecules, retention time reproducibility, and noise from abundant plasma constituents. The system may automatically sort analytes into specialized assay clusters designed to maximize sensitivity and reproducibility while reducing total run time. Additionally, patterns of recurring plasma matrix interference—potentially caused by high-abundance proteins or lipids—are confirmed across thousands of samples, offering opportunities to automate data analysis further.

Overall, this two-phase (discovery→validation) methodology greatly broadens the catalog of plasma-detectable proteins and small molecules, while imposing strict standards for reproducibility and quantitative reliability. In some embodiments, the validated repository derived from these iterative procedures supports research or clinical applications requiring sub-ng/L detection sensitivity and reproducibility coefficients of variation below about 10%. By delivering both an extensive coverage of potential biomarkers and fine-tuned detection parameters, this two-phase pipeline furnishes a robust platform for high-throughput plasma analyses across proteomic, metabolomic, and lipidomic domains.

Accordingly, in some embodiments, the entire system may also incorporate AI-driven learning or peak-picking algorithms that rely on tens or hundreds of thousands of curated spectral files, further minimizing technical variation and enhancing the accuracy of final reproducibility metrics. Through these iterative improvements, the pipeline's validated module ultimately forms a dynamic, continuously updating platform that surpasses conventional single-phase protein or small-molecule databases in consistency and scale, thus bridging recognized gaps in prior proteomics references.

By integrating advanced functionalization strategies, tunable porosity, and exceptional reusability, the magnetic nanoparticles developed in this invention address key challenges in plasma proteomics by combining advanced material science with precise functionalization. Their scalability, reusability, and exceptional performance metrics make them indispensable for both research and clinical applications, paving the way for breakthroughs in disease diagnostics and personalized healthcare.

### V. Plasma Proteome Variation and Genetic Determinants

Numerous studies emphasize the complexity of plasma proteomics due to the extensive dynamic range of protein concentrations and the inherent variability of human samples. For instance, Anderson & Anderson (2002, Mol. Cell. Proteomics 1:845-867) highlight the challenges in mapping a significant fraction of the plasma proteome—particularly low-abundance proteins—while large-scale collaborations (e.g., the HUPO Plasma Proteome Project) recognized that robust coverage and reproducibility had yet to be consistently achieved in broad population studies. Moreover, attempts to correlate plasma protein expression with demographic and physiological parameters (e.g., age, gender, BMI) or genetic polymorphisms frequently encountered assay limitations, partial coverage, and high inter-run variance, as noted in early references such as Ommen et al. (2012) or various single-lab pilot analyses focusing on limited sets of candidate proteins.

In some embodiments, the disclosed pipeline enables ultra-deep plasma proteomics aimed at identifying how protein expression levels correlate with demographic and physiological variables such as age, gender, or Body Mass Index (BMI). While prior research (e.g., Mann et al., ref. 74) suggests that a substantial portion of the plasma proteome varies with these parameters, widespread application in large cohorts has historically been constrained by assay limitations, including insufficient sensitivity and inconsistent reproducibility. Here, the pipeline's high-sensitivity detection capacity facilitates the measurement of plasma proteins spanning multiple orders of magnitude in abundance, revealing shifts in proteins linked to inflammation, extracellular matrix remodeling, lipid metabolism, and coagulation cascades.

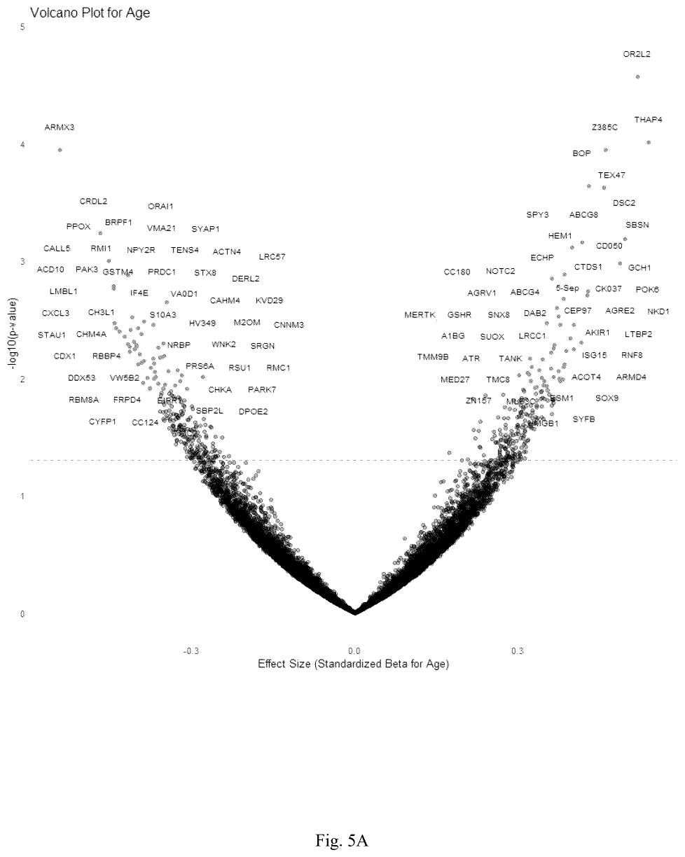

In certain embodiments, data derived from these analyses (e.g., FIG. 5) highlight BMI-associated protein signatures, including TRIB3 (a regulator of insulin resistance), INHBE (related to fat distribution), ERBB4 (involved in energy expenditure), and LEP (leptin)—reinforcing leptin's recognized role in weight homeostasis. By capturing these low- and moderate-abundance proteins in plasma, the invention allows for the establishment of metabolic state biomarkers with relevance to early disease detection and personalized health monitoring. Such findings offer robust molecular signatures to guide future biomarker discovery or risk stratification, reflecting a potential “biological clock” concept for metabolic health.

In further embodiments, the pipeline supports genetic-proteomic association studies, sometimes termed pQTL (proteomic quantitative trait loci) analysis, where the identified proteins are cross-correlated with genomic data to uncover cis- or trans-regulatory effects. Incorporating reference sequences or GWAS findings, the system can illuminate causal relationships between genetic variants, protein-level alterations, and eventual disease predispositions. Although theoretical frameworks for linking proteomic variation with genotype have existed, practical implementation has been hampered by incomplete or inconsistent coverage across large-scale plasma samples. Here, the pipeline's reproducibility (coefficient of variation <10%) and extensive dynamic range serve to mitigate these challenges, enabling robust pQTL exploration.

In some embodiments, the system quantifies thousands of plasma proteins with minimal technical noise, facilitating detailed modeling of age- and BMI-related phenotypes. The results—demonstrated, for instance, through data sets or tables correlating particular proteins with demographic strata—reinforce the pipeline's potential in precision medicine, where individualized protein expression patterns can inform therapeutic targeting or early detection strategies. By supporting this expanded coverage of plasma proteins and effectively linking them to metabolic and genetic determinants, the invention extends beyond traditional proteomics approaches, enabling broad insights into human health at the molecular level.

### VI. Dried Blood Spot (DBS) Sample Analysis

In some embodiments, the disclosed pipeline is adapted for dried blood spot (DBS) collection, commonly chosen for at-home sampling due to ease of handling, low storage requirements, and cost-effective transportation. Literature acknowledges DBS as an established technique for small-molecule assays (e.g., Evans et al., AAPS J. 2015 March; 17(2):292-300.) and certain immunoassays, but proteomics efforts can be hindered by epitope degradation or extensive peptide release from lysed erythrocytes during ambient storage (Abeline et al., Nat Commun. 2023 Apr. 3; 14(1):1851). Consequently, many existing workflows either limit the range of analytes tested or require specialized preservation methods.

In contrast, certain embodiments herein deploy a proprietary optimization strategy to detect protein targets resilient to proteolysis and chemical modifications. High-abundance or unstable proteins that are released during prolonged room-temperature storage can, in some embodiments, be explicitly excluded, ensuring that the final dataset retains only clinically relevant analytes. This design confers high sensitivity and specificity even under extended ambient storage, circumventing the pitfalls typically seen in epitope-based approaches.

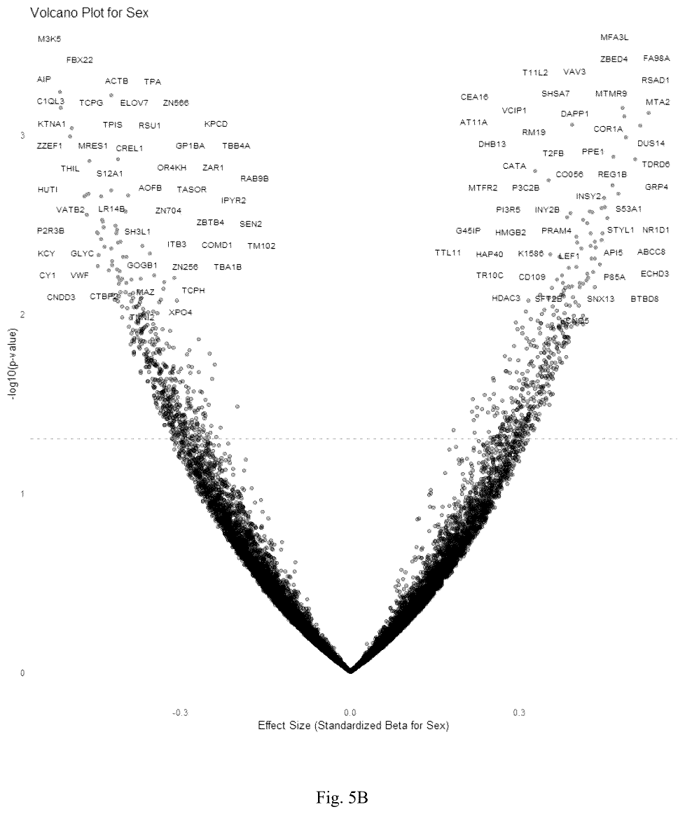

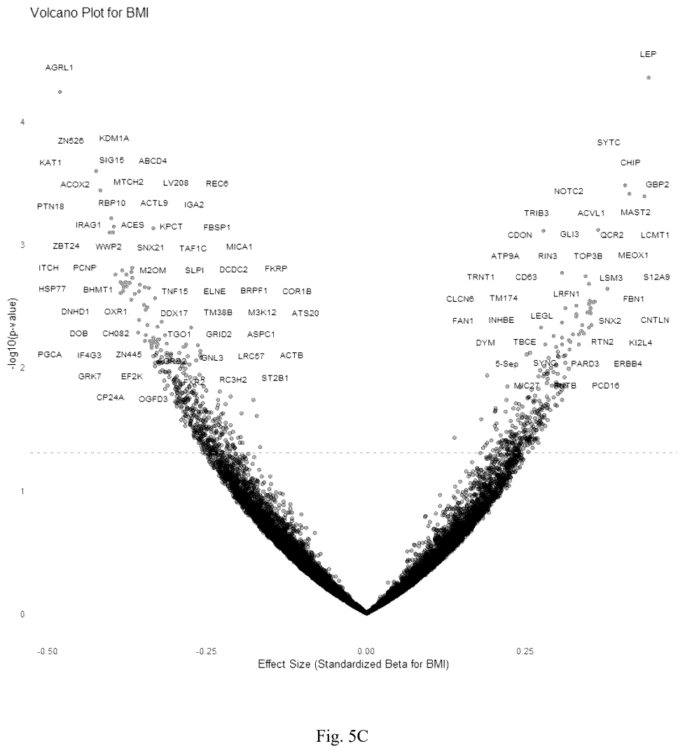

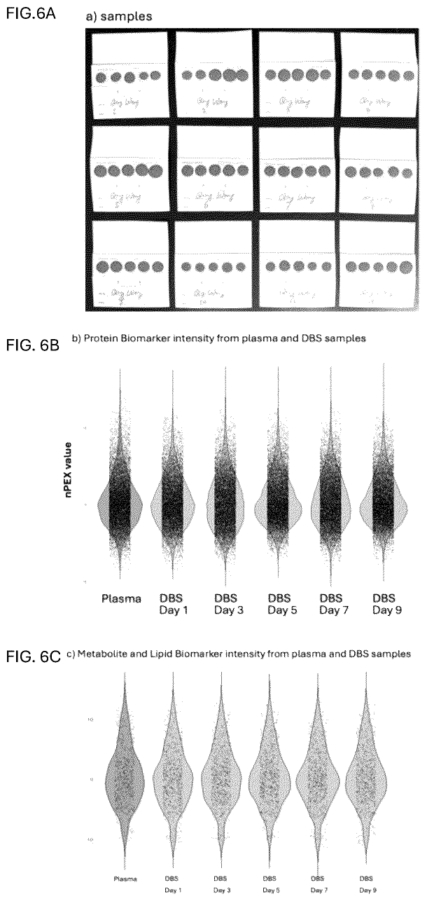

To illustrate the pipeline's capacity to maintain performance across varied storage durations, a series of DBS samples were collected and stored at ambient temperature (e.g., 1 to 12 days) in standard mailing envelopes, simulating realistic transport. These samples were processed using a proprietary in-house method and analyzed for proteins and small molecules with the integrated workflow. In certain embodiments, FIG. 6 depicts the timeline or layout of this DBS storage experiment, while subsequent data analyses confirm the system's robustness in measuring thousands of proteins from each DBS sample.

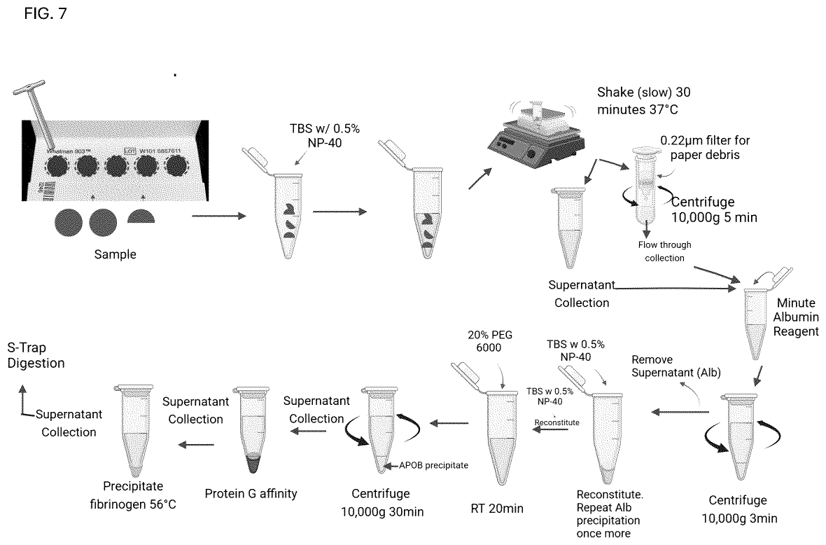

Through this pipeline, over 10,000 proteins were systematically quantified from DBS samples. Approximately 2-3% of these proteins exhibited consistent temporal variations (e.g., about 340 showing a steady increase and 279 a continuous decrease) over multiple storage intervals (1, 3, 5, 7, 9 days). Additionally, about 46.96% of proteins maintained a coefficient of variation (CV) below 25% across all DBS samples from a single individual-underscoring, in some embodiments, the approach's stability even under air and room-temperature exposure. Furthermore, comparisons with fresh plasma samples from the same individual suggest close alignment between DBS-derived proteomic profiles and standard plasma proteomes (see FIG. 7), thereby validating the pipeline's capability in bridging dried and conventional sample collection methods.

In certain embodiments, a normalization reference dataset is compiled, capturing all detected proteins from a 12-day storage window for a given individual. By integrating a mailing timestamp or equivalent shipping metadata, subsequent analyses can adjust for time-dependent changes in protein abundance, ensuring that the measured analytes accurately reflect the original clinical status at the time of DBS sample collection. Meanwhile, additional discovery-mode runs using a timsTOF or equivalent high-resolution platform identified about 5,781 proteins from DBS, including at least 133 proteins not previously observed in fresh plasma reference datasets—thus broadening the scope of detectable analytes beyond existing repositories (e.g., a 17,328-protein database).

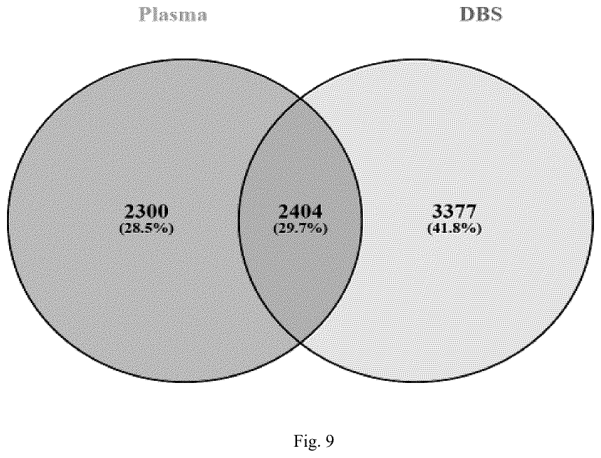

DBS also proves inherently robust for metabolomics and lipid assays. In further embodiments, the pipeline detects ˜1,395 metabolites and 762 lipids from DBS samples, facilitating a complete multi-omics workflow across proteins, lipids, and small molecules. Observations in these trials suggest that the levels of numerous small molecules remain sufficiently stable over multiple days (see also FIG. 9), reinforcing the view that DBS can effectively support longitudinal or remote-sample studies. By enabling advanced multi-omics analyses from minimal sample volumes stored at ambient temperature, the pipeline enables new avenues for remote patient monitoring, large-scale epidemiological research, and globally distributed healthcare applications. In some embodiments, this capacity to collect robust proteomics, metabolomics, and lipidomics data-despite varied sample collection and shipping conditions-redefines clinical proteomics by offering a consistent, high-quality multi-omics dataset from diverse logistical scenarios.

### VII. Automated Machine-Learning Peak Analysis and Data Processing

Historically, automated peak picking and quantification in proteomics has relied on heuristic or rule-based algorithms that often fail when faced with large-scale plasma datasets or highly complex chromatographic profiles. Several works, including Neely et al. (J Proteome Res. 2023 Mar. 3; 22(3):681-696.) and Mann et al. (Cell Syst. 2021 Aug. 18; 12(8):759-770.), describe how machine-learning (ML) methods can enhance peak detection and reduce manual labor. However, these solutions typically address only a subset of the proteome or do not incorporate a robust manual-curation backbone that ensures consistent detection across the diverse conditions and disease states encountered in clinical proteomics. Additionally, prior art seldom unifies manual validation data with large-scale ML training to achieve both high throughput and low false positive rates in data from tens of thousands of peptides.

In some embodiments, the present invention employs a comprehensive bioinformatic pipeline termed “CompletePeaking” to support clinical proteomics and metabolomics data processing. CompletePeaking integrates manual curation of proteomics data with an automated machine-learning module to yield accurate identification and quantification of peptides (and other analytes) in complex samples. For instance, it may incorporate FIG. 1 to illustrate the pipeline steps, from manual labeling of reference spectra to final ML-based peak selection.

Initially, a manual validation stage is used to review chromatograms and spectra for each peptide transition. This process typically involves close examination of signal-to-noise ratio (SNR), intensity, retention-time coelution, and library dot product scores—quantifying the similarity between observed signals and reference spectral intensities. In certain embodiments, peaks that exhibit robust coelution and high dot product scores are tagged as “good” peaks, forming the training dataset for the subsequent machine-learning model. Because this manual labeling process is conducted across diverse clinical samples (e.g., advanced cancer plasma, cardiovascular disease samples, neurodegenerative disease samples, and autoimmune disease plasma), a broad array of potential noise patterns and coelution behaviors is captured.

In some embodiments, years of manual peak validation across thousands of samples yields an extensive curated data repository. This repository is used to train an ML classifier, such as one based on the XGBoost algorithm, which incorporates multiple features derived from chromatogram analysis. These features may include coelution count (the number of transitions aligned within a set tolerance window, e.g., ±0.3 minutes), overall SNR, dot product values, shape correlation, and raw intensity levels. A particularly high coelution count may enhance confidence in the validity of a peak, while other features can discount signals suspected to originate from chemical noise or co-eluting contaminants.

The machine-learning model is typically optimized through cross-validation, ensuring it generalizes across different biological matrices and disease states. In certain embodiments, the validated pipeline is assessed using a dataset of more than 15,000 peptides spanning varied clinical samples (e.g., advanced cancer, inflammatory diseases), confirming the model's ability to reliably identify peaks under heterogeneous conditions. By merging manual curation with a data-driven ML approach, the pipeline addresses a key limitation in conventional proteomics workflows, wherein pure algorithmic methods may produce false positives or low-intensity peaks with uncertain biological relevance.

In some embodiments, after training and validation, the model is deployed to unseen data for automated peak picking. Postprocessing steps include selecting the highest-confidence peaks for each peptide sequence and normalizing the resulting peak area data across distinct sample clusters. Because the pipeline is often applied to large, complex datasets (e.g., tens of thousands of peptides, multiple scanning methods, varied retention-time scheduling), normalization can be performed separately for each cluster, leading to the derivation of a “Multi-point Normalized Protein expression” (MNPX) value or similar measure. This MNPX factor allows cross-sample comparisons of protein abundance while mitigating run-to-run or instrument-based variability.

In further embodiments, Example 2 detail the differences between manual validation alone and the combined manual-ML approach, demonstrating improved throughput and consistent low false-positive rates. By adopting a robust manual-labeled training set—sometimes accumulated over multi-year curation—CompletePeaking capitalizes on a diverse set of patterns relating to co-elution artifacts, chemical noise, or offset retention times. This synergy ensures sub-10% coefficient of variation in repeated quantitations for many target peptides and proteins, thus advancing the pipeline's utility for clinical proteomics.

Overall, in some embodiments, the combination of manual curation and automated ML not only expands the scale at which proteomic analyses can be undertaken but also elevates the reliability of peak picking for large-scale, high-throughput clinical or translational research projects. By mapping years of curated data to an advanced classification model, the invention bridges the gap between labor-intensive expert validation and purely algorithmic solutions, delivering a more consistent, high-accuracy, and scalable platform for multi-omics data processing in plasma or other clinical samples.

### Example 1: Complete360: a Multi-Omics Liquid Biopsy Platform with Attomole Sensitivity and High Reproducibility

### I. Introduction

Blood is one of the most accessible clinical samples for liquid biopsy, making it invaluable for molecular diagnostics in human disease. Several methods have been previously reported for detecting and quantifying disease biomarkers, particularly nucleic acids in the context of human cancers, and some of these methods have successfully led to commercial applications in clinical settings¹⁻³. Since cancers are often associated with mutations, translocations, and other genetic alterations, these changes can typically be measured from circulating tumor DNA in the blood⁴. However, many human diseases—such as cardiovascular, neurodegenerative, and autoimmune disorders—do not involve DNA mutations and cannot be assessed through sequencing methods. Additionally, genetic predispositions to cancers, once measured in a person to assess cancer risks, the subsequent measurement provides no additional information for clinical intervention, which limits the clinical demand for sequencing technologies aimed at detecting nucleic acid changes in disease diagnostics.

In contrast, proteins and small molecules (metabolites and lipids) in human blood serve as excellent surrogates that reflect real-time changes in health status^{5, 6}. Virtually all human diseases can be monitored through variations in protein or small molecule surrogate levels in blood, and most clinical tests are indeed measuring proteins or small molecules⁷. However, accurately measuring these molecules in blood has proven to be a significant challenge, limiting their clinical applications.

Traditional methods for measuring proteins and metabolites in blood can be categorized into two main approaches. The first is the affinity-based method. The second approach involves mass spectrometry, which can be performed in either targeted or untargeted modes. While untargeted mass spectrometry provides a broader range of identified proteins, it generally lacks sensitivity for blood proteomics. The advent of advanced mass spectrometers, such as the timsTOF HT from Bruker and Astral from ThermoFisher, in combination with low abundant blood protein enrichment strategies, has allowed researchers to detect ˜6,000 proteins from plasma samples^{8, 9}. In prior work, highly sensitive detection of specific mutated forms of proteins is achieved through a modified targeted approach⁶, demonstrating high sensitivity after a series of parameter optimizations aimed at ultra-low abundance detection, including neoantigens¹⁰⁻¹³.

It was hypothesized that, through recursive optimization of detection parameters for each individual protein target and the compilation of these optimized protocols into a unified assay, accurate detection and quantification across the human plasma proteome could be achieved, meeting the sensitivity and reproducibility standards required for clinical applications. By refining liquid chromatography-mass spectrometry (LC-MS) parameters and developing customized sample preparation methods specific to each protein target, unprecedented sensitivity was reached using a targeted mass spectrometry assay. The Complete360® platform—a multi-omics system designed to support both research and clinical diagnostics—was developed to detect over 10,000 human proteins and more than 2,000 metabolites and lipids. This platform executes a fully optimized and automated workflow integrating sample preparation, mass spectrometry method clusters, and data analysis. Complete360® addresses the limitations of traditional mass spectrometry by substantially improving the reproducibility and detectability of proteins, metabolites, and lipids frequently missed by conventional technologies. It generates more accurate multi-omics datasets by capturing larger biological differences between diseased and normal samples, thereby enhancing its suitability for clinical diagnostic use.

### II. Results

### Complete360® Platform: Integrating Extensive Data, Manual Curation, and Artificial Intelligence (AI)

The Complete360® platform comprises of 1) CompleteBank development for targeted assay panel construction, 2) extraction of plasma proteins, metabolites, and lipids from blood samples, 3) mass spectrometric analysis, and 4) data analysis using CompletePeaking Algorithm for report (FIG. 1A).

Applicants developed a comprehensive database, termed CompleteBank, structured in two phases: CompleteBank-Discovered and CompleteBank-Validated. In the first phase, Applicants created a discovery database encompassing 17,328 proteins and 2,927 metabolites/lipids observed in various human blood samples as described in Table 2. This module, termed CompleteBank-Discovered (FIG. 1A), serves as a repository for preliminary detection parameters of blood signatures, laying the groundwork for further validation. To assess the depth and uniqueness of CompleteBank-Discovered, current protein list is compared with the recently published Human Proteome Project (HPP) database¹⁴. Remarkably, 536 proteins are identified from the PE2-5 groups, marking the first-ever detection of these proteins by mass spectrometry, representing approximately 2.74% of the human proteome. This achievement is particularly significant given that these proteins were detected in blood, where protein identification is inherently more challenging. Curent findings not only expand the known repertoire of detectable human proteins but also highlight the completeness and robustness of Applicants' foundational database.

The second phase focused on rigorous validation to ensure the detectability and reliability of the proteins and metabolites identified in the discovery phase. This involved a minimum of five iterative rounds of optimization for each target molecule on liquid chromatographic separation, mass spectrometric analysis, and transition selection, aimed at maximizing detection sensitivity and specificity. After each experimental round, data underwent meticulous curation. Through this process, targeted mass spectrometric assays are curated using dynamic Selected Reaction Monitoring (dSRM) corresponding to 10,598 blood proteins and 2,157 small molecules, which collectively form CompleteBank-Validated module (FIG. 1A). This module has been developed through repeated optimizations across over 9,000 individual LC-MS/MS runs on a TOF mass spectrometer and over 1,000,000 runs on triple quadrupole (QqQ) mass spectrometers. Over 600,000 QqQ raw spectrum files were manually reviewed to ensure data quality. The CompleteBank-Validated module is enriched with precise detection parameters, including optimization on retention times, m/z values for MS1 and MS2 fragments, collision energies, source parameters and blood-specific noises as well as target-specific noises. It also incorporates sample preparation parameters, such as optimal sample preparation buffer systems for each individual sample type and target, and protection peptides used for each target through Applicants' MaxRec technology¹².

To evaluate the sources from the optimized CompleteBank-Validated proteins, the UniProt tissue annotation database (UP_TISSUE) was used, and it is found that the validated blood proteins developed in Complete360® platform came from all major human tissues or organs (Table 3, FIG. 1B). Additionally, subcellular localization analysis revealed the following distribution: 34.5% cytoplasmic, 11.8% secreted, 9% cytoskeletal, 8.2% mitochondrial, 8.0% endoplasmic reticulum, 7.8% cell projection, 6.5% Golgi apparatus, 4.7% cytoplasmic vesicle, and 4.4% endosomal proteins, etc (FIG. 1C); and 2,208 small molecules, where there are 762 polar metabolites from different classes (organic acids, amino acids, nucleotides, carbohydrates, etc.), covering most of the human metabolic, drug and disease pathways, and 1,395 lipid species across more than 24 (sub) classes (including aceylCarn, Bile acids, CE, CL, DAG, DE, DG, FFA, HexCer, LPC, LPE, LPG, LPI, MAG, PA, PC, PE, PG, PI, PL, PS, PIP, SIP, SM, SP, SPN, TAG, TG) 15-19 (FIG. 1C).

TABLE 3
Low-Abundance Proteins on Complete360 target list Tissue Expression Annotation
of Complete360 Proteins (10227 out of 10598 proteins are annotated)

| | Number of | % of Proteins in | | |
|---|---|---|---|---|
| Tissue | Proteins | the Panel | P-Value | Benjamini |
| Brain | 4980 | 46.4 | 2.00E−26 | 1.70E−24 |
| Liver | 3899 | 36.4 | 9.30E−249 | 6.90E−246 |
| Cervix | 3436 | 32.0 | 2.30E−97 | 8.40E−95 |
| carcinoma | | | | |
| Erythroleukemia | 3041 | 28.4 | 3.30E−94 | 8.20E−92 |
| Placenta | 2397 | 22.4 | 7.10E−40 | 7.50E−38 |
| Leukemic T-cell | 1709 | 15.9 | 3.50E−58 | 4.30E−56 |
| Lung | 1595 | 14.9 | 5.20E−06 | 1.40E−04 |
| Uterus | 1208 | 11.3 | 1.50E−11 | 7.40E−10 |
| Skin | 1138 | 10.6 | 3.30E−13 | 1.90E−11 |
| Kidney | 994 | 9.3 | 5.30E−06 | 1.40E−04 |
| Colon | 750 | 7.0 | 4.70E−04 | 7.30E−03 |
| Cerebellum | 707 | 6.6 | 3.70E−05 | 7.60E−04 |
| Eye | 642 | 6.0 | 4.40E−06 | 1.30E−04 |
| Pancreas | 598 | 5.6 | 1.10E−05 | 2.60E−04 |
| Amygdala | 562 | 5.2 | 1.60E−03 | 2.00E−02 |
| Muscle | 556 | 5.2 | 4.60E−13 | 2.50E−11 |
| Trachea | 553 | 5.2 | 9.70E−06 | 2.50E−04 |
| Spleen | 532 | 5.0 | 2.90E−03 | 3.60E−02 |
| Tongue | 523 | 4.9 | 3.10E−08 | 1.10E−06 |
| Fetal brain | 517 | 4.8 | 1.00E−03 | 1.40E−02 |
| Ovary | 510 | 4.8 | 3.80E−03 | 4.50E−02 |
| Platelet | 496 | 4.6 | 8.00E−90 | 1.50E−87 |
| Hippocampus | 491 | 4.6 | 5.40E−03 | 6.10E−02 |
| Thymus | 469 | 4.4 | 2.00E−04 | 3.40E−03 |
| Blood | 465 | 4.3 | 1.90E−04 | 3.40E−03 |
| Bone marrow | 454 | 4.2 | 7.90E−04 | 1.10E−02 |
| Lymph | 448 | 4.2 | 1.50E−09 | 5.80E−08 |
| Heart | 425 | 4.0 | 1.30E−05 | 3.00E−04 |
| Skeletal muscle | 419 | 3.9 | 1.70E−06 | 5.40E−05 |
| Thalamus | 389 | 3.6 | 6.90E−04 | 1.00E−02 |
| Embryonic | 388 | 3.6 | 4.40E−11 | 2.10E−09 |
| kidney | | | | |
| Colon | 387 | 3.6 | 5.60E−11 | 2.40E−09 |
| carcinoma | | | | |
| Prostate | 384 | 3.6 | 1.50E−02 | 1.40E−01 |
| Cervix | 319 | 3.0 | 2.10E−04 | 3.60E−03 |
| Mammary gland | 296 | 2.8 | 4.90E−03 | 5.60E−02 |
| Lymphoblast | 292 | 2.7 | 2.00E−26 | 1.70E−24 |
| Plasma | 286 | 2.7 | 1.10E−60 | 1.70E−58 |
| Melanoma | 274 | 2.6 | 4.00E−09 | 1.50E−07 |
| Umbilical cord | 241 | 2.2 | 2.60E−04 | 4.10E−03 |
| blood | | | | |
| Embryo | 234 | 2.2 | 3.60E−02 | 2.90E−01 |
| T-cell | 210 | 2.0 | 6.50E−08 | 2.20E−06 |
| Fetal brain | 187 | 1.7 | 2.90E−21 | 2.00E−19 |
| cortex | | | | |
| Cajal-Retzius | 179 | 1.7 | 2.90E−26 | 2.20E−24 |
| cell | | | | |
| Hepatoma | 175 | 1.6 | 1.20E−04 | 2.20E−03 |
| Stomach | 159 | 1.5 | 2.20E−02 | 1.90E−01 |
| Fetal liver | 158 | 1.5 | 3.70E−05 | 7.60E−04 |
| Urinary bladder | 157 | 1.5 | 3.80E−03 | 4.50E−02 |
| Caudate nucleus | 145 | 1.4 | 7.70E−03 | 8.10E−02 |
| Adrenal gland | 143 | 1.3 | 1.30E−02 | 1.20E−01 |
| PNS | 133 | 1.2 | 1.00E−02 | 9.80E−02 |
| Neuroblastoma | 129 | 1.2 | 4.70E−02 | 3.60E−01 |
| Ovarian | 127 | 1.2 | 1.60E−03 | 2.10E−02 |
| carcinoma | | | | |
| B-cell | 120 | 1.1 | 9.00E−14 | 5.60E−12 |
| lymphoma | | | | |
| Leukocyte | 118 | 1.1 | 1.50E−03 | 2.00E−02 |
| Fetal kidney | 117 | 1.1 | 1.90E−02 | 1.70E−01 |
| Synovium | 116 | 1.1 | 2.10E−06 | 6.10E−05 |
| Lymph node | 114 | 1.1 | 1.20E−02 | 1.20E−01 |
| Keratinocyte | 103 | 1.0 | 2.00E−06 | 6.10E−05 |
| Fibroblast | 103 | 1.0 | 1.20E−04 | 2.20E−03 |
| Corpus | 92 | 0.9 | 3.10E−02 | 2.50E−01 |
| callosum | | | | |
| Pituitary | 90 | 0.8 | 2.10E−02 | 1.80E−01 |
| Hypothalamus | 87 | 0.8 | 7.90E−02 | 5.40E−01 |
| Mammary | 83 | 0.8 | 2.90E−05 | 6.60E−04 |
| carcinoma | | | | |
| Endometrium | 70 | 0.7 | 6.00E−03 | 6.70E−02 |
| Breast | 69 | 0.6 | 5.80E−02 | 4.20E−01 |
| Aorta | 66 | 0.6 | 1.00E−02 | 9.90E−02 |
| Lung carcinoma | 65 | 0.6 | 5.40E−02 | 4.00E−01 |
| Colon | 63 | 0.6 | 2.50E−04 | 4.00E−03 |
| adenocarcinoma | | | | |
| Lymphocyte | 61 | 0.6 | 3.80E−02 | 3.10E−01 |
| Saliva | 56 | 0.5 | 4.90E−03 | 5.60E−02 |
| Bone | 54 | 0.5 | 8.10E−03 | 8.40E−02 |
| Leukemia | 52 | 0.5 | 8.80E−03 | 9.00E−02 |
| Cerebrospinal | 49 | 0.5 | 1.40E−10 | 5.80E−09 |
| fluid | | | | |
| Cord blood | 49 | 0.5 | 1.20E−02 | 1.20E−01 |
| Prostate cancer | 45 | 0.4 | 5.80E−04 | 8.60E−03 |
| Renal cell | 43 | 0.4 | 8.90E−02 | 6.00E−01 |
| carcinoma | | | | |
| Colon | 42 | 0.4 | 2.90E−03 | 3.60E−02 |
| endothelium | | | | |
| Endothelial cell | 42 | 0.4 | 3.00E−02 | 2.50E−01 |
| Lymphoma | 42 | 0.4 | 4.30E−02 | 3.40E−01 |
| Foreskin | 38 | 0.4 | 9.60E−03 | 9.50E−02 |
| Human small | 37 | 0.3 | 5.50E−04 | 8.30E−03 |
| intestine | | | | |
| Umbilical vein | 37 | 0.3 | 7.70E−03 | 8.10E−02 |
| endothelial cell | | | | |
| Chondrosarcoma | 37 | 0.3 | 1.30E−02 | 1.20E−01 |
| Lung fibroblast | 34 | 0.3 | 4.40E−02 | 3.40E−01 |
| Peripheral | 34 | 0.3 | 4.40E−02 | 3.40E−01 |
| Nervous System | | | | |
| Osteosarcoma | 33 | 0.3 | 5.60E−02 | 4.10E−01 |
| Milk | 32 | 0.3 | 9.90E−05 | 2.00E−03 |
| Uterine | 32 | 0.3 | 6.50E−03 | 7.10E−02 |
| endothelium | | | | |
| Endometrial | 32 | 0.3 | 7.00E−02 | 4.90E−01 |
| tumor | | | | |
| Neutrophil | 29 | 0.3 | 1.20E−04 | 2.20E−03 |
| Umbilical vein | 29 | 0.3 | 9.40E−03 | 9.50E−02 |
| Aortic | 28 | 0.3 | 1.90E−04 | 3.40E−03 |
| endothelium | | | | |
| Adipocyte | 28 | 0.3 | 1.50E−03 | 2.00E−02 |
| Erythrocyte | 26 | 0.2 | 3.20E−05 | 7.00E−04 |
| Skeletal Muscle | 26 | 0.2 | 2.50E−02 | 2.10E−01 |
| Urine | 25 | 0.2 | 2.00E−03 | 2.50E−02 |
| Epithelium | 24 | 0.2 | 1.20E−03 | 1.60E−02 |
| Whole blood | 24 | 0.2 | 7.00E−03 | 7.50E−02 |
| Aorta | 24 | 0.2 | 7.10E−02 | 4.90E−01 |
| endothelial cell | | | | |
| Bile | 23 | 0.2 | 2.60E−05 | 6.10E−04 |
| Serum | 21 | 0.2 | 4.10E−04 | 6.40E−03 |
| Retinal pigment | 21 | 0.2 | 2.10E−02 | 1.80E−01 |
| epithelium | | | | |
| Stomach | 20 | 0.2 | 1.50E−02 | 1.30E−01 |
| mucosa | | | | |
| Cartilage | 20 | 0.2 | 2.90E−02 | 2.50E−01 |
| Mesangial cell | 20 | 0.2 | 5.30E−02 | 4.00E−01 |
| Tear | 19 | 0.2 | 7.20E−02 | 4.90E−01 |
| Chondrocyte | 18 | 0.2 | 5.70E−02 | 4.20E−01 |
| Lens epithelium | 15 | 0.1 | 8.50E−02 | 5.80E−01 |
| Astrocytoma | 12 | 0.1 | 6.90E−02 | 4.90E−01 |
| Neuroepithelium | 11 | 0.1 | 4.60E−02 | 3.50E−01 |
| Vascular | 10 | 0.1 | 7.00E−02 | 4.90E−01 |
| smooth muscle | | | | |

The method development phase is extensively relied on manual curation, which proved indispensable for enhancing the quality and reliability of current database. This meticulous process allowed the researchers to fine-tune detection parameters. Over the course of approximately six years, Applicants iteratively optimized these parameters while documenting more than 9,000 blood samples. This process enabled high sensitivity for each target detected via mass spectrometry. With these large manually curated datasets in hand, an AI-based learning and peak-picking system is developed and named as CompletePeaking (FIG. 1A). This system performs data analysis for each sample using comprehensive pattern-recognition algorithms to improve the identification of precise peaks for each target, with particular emphasis on determining the optimal transition(s) 20. The manually curated spectral files serve as the training set for the CompletePeaking algorithm. With CompletePeaking, the detection of all analytes has been consolidated into a suite of mass spectrometry assay clusters designed to maximize both sensitivity and reproducibility while minimizing runtime. The development of these assays incorporated several key considerations, including separation of high- and low-abundance molecules, resolution of co-eluted targets, ensure retention time reproducibility, management of co-eluting noise analytes, and minimizing Run Time, which detailed in method session.

Worthy of highlighting, as previously mentioned in management of co-eluting noise analytes, when analyzing blood samples, similar noise patterns are consistently observed across different specimens. These patterns likely originate from common high-abundance proteins or blood matrix components, such as lipids and other prevalent substances^{21, 22}. These elements, while abundant, are known to limit the depth of analysis in blood proteomics and are considered detrimental to proteomic assays²³. However, their consistent reproducibility, after being confirmed from thousands of samples, provides an opportunity to enhance data analysis automation. This consistency enables more precise peak-picking for peptide and molecule targets without the need for spiking in exogenous standards.

### Dynamic Range and Detectability of Extremely Low-Abundance Blood Proteins

Proteins in blood exhibit a vast dynamic range of concentrations, which presents a significant challenge in achieving the desired analytical depth for clinical proteomics assays. To evaluate the dynamic range of detectability provided by the Complete360® platform, a small panel of well-documented plasma proteins with known concentrations are selected. Current findings reveal that the Complete360® pipeline enables the detection of plasma proteins within a remarkable concentration range—from ˜10 μg/mL to ˜100 μg/mL. This dynamic range encompasses the physiological plasma concentrations of most known plasma proteins (FIG. 2).

One of the most challenging aspects of mass spectrometry-based detection of blood proteins is sensitivity, which depends on factors such as co-elution dynamics, contamination, and ion suppression. Detecting low-abundance protein targets has been a critical area of interest. Through repeated optimizations, Applicants have demonstrated the ability to push the detection limits of a standard QqQ instrument to unprecedented levels at pg/mL (FIG. 2), enabling the detection of extremely low-abundance targets^{6, 10-13, 24}. To further evaluate the detectability of Complete360® for extremely low-abundance plasma proteins, assays are conducted using pooled plasma samples from healthy individuals described in previous studies⁶. The proteins detected are annotated with plasma concentration data from the Human Proteome Project (HPP) database and commercial assay providers. Notably, the lowest annotated protein detected by Complete360® is identified at a concentration of 3.5 pg/mL (Table 4). Furthermore, numerous proteins in the sub-10 μg/mL range exhibit robust peak profiles on the Complete360® pipeline, underscoring its capability to detect even lower-abundance targets. For example, the protein Isocitrate dehydrogenase [NAD] subunit beta, mitochondrial (Uniprot ID: O43837) with a reported plasma concentration as 8.3 pg/mL was detected in different plasma samples at high abundance, covering various disease conditions (data not shown). Despite its reported pg/mL level concentration, the signal intensity for this protein observed from Complete360® was strong that it remained detectable after a 1:1000 dilution (data not shown). These findings also indicate that Complete360® may achieve excellent detection performance even with further reduced sample input volumes. This feature has the potential to enable novel applications, such as in-home sample collection. Beyond Isocitrate dehydrogenase, several other low-abundance proteins demonstrated similarly strong intensity, including Leukocyte cell-derived chemotaxin 1 (UniprotID: 075829), NADH dehydrogenase [ubiquinone] flavoprotein 2, mitochondrial (UniprotID: P19404), Calretinin (UniprotID: P22676), and Methionine aminopeptidase 1 (UniprotID: P53582), etc.

TABLE 4
Low-Abundance Proteins on current platform target list

| | | | | Conc |
|---|---|---|---|---|
| | | | | (attomole/ |
| | | | | ML)HP |
| Protein | Protein Name | Length | Mass | PP |
| Q63HN8 | E3 ubiquitin-protein ligase RNF213 (EC | 5207 | 591407 | 5.92 |
| | 2.3.2.27) (EC 3.6.4.-) (ALK lymphoma | | | |
| | oligomerization partner on chromosome 17) | | | |
| | (E3 ubiquitin-lipopolysaccharide ligase | | | |
| | RNF213) (EC 2.3.2.-) (Mysterin) (RING | | | |
| | finger protein 213) | | | |
| P46939 | Utrophin (Dystrophin-related protein 1) | 3433 | 394466 | 10.65 |
| | (DRP-1) | | | |
| Q8TDW7 | Protocadherin Fat 3 (hFat3) (Cadherin family | 4557 | 501978 | 9.36 |
| | member 15) (FAT tumor suppressor | | | |
| | homolog 3) | | | |
| Q9UBC9 | Small proline-rich protein 3 (22 kDa | 169 | 18154 | 286.44 |
| | pancornulin) (Cornifin beta) (Esophagin) | | | |
| Q5VYK3 | Proteasome adapter and scaffold protein | 1845 | 204291 | 26.43 |
| | ECM29 (Ecm29 proteasome adapter and | | | |
| | scaffold) (Proteasome-associated protein | | | |
| | ECM29 homolog) | | | |
| Q9UPQO | LIM and calponin homology domains- | 1083 | 121867 | 45.95 |
| | containing protein 1 | | | |
| P48634 | Protein PRRC2A (HLA-B-associated | 2157 | 228863 | 24.47 |
| | transcript 2) (Large proline-rich protein | | | |
| | BAT2) (Proline-rich and coiled-coil- | | | |
| | containing protein 2A) (Protein G2) | | | |
| O14639 | Actin-binding LIM protein 1 (abLIM-1) | 778 | 87688 | 68.42 |
| | (Actin-binding LIM protein family member | | | |
| | 1) (Actin-binding double zinc finger protein) | | | |
| | (LIMAB1) (Limatin) | | | |
| Q02388 | Collagen alpha-1(VII) chain (Long-chain | 2944 | 295220 | 20.32 |
| | collagen) (LC collagen) | | | |
| Q9BTW9 | Tubulin-specific chaperone D (Beta-tubulin | 1192 | 132600 | 46.76 |
| | cofactor D) (tfcD) (SSD-1) (Tubulin-folding | | | |
| | cofactor D) | | | |
| Q15042 | Rab3 GTPase-activating protein catalytic | 981 | 110524 | 57.00 |
| | subunit (RAB3 GTPase-activating protein | | | |
| | 130 kDa subunit) (Rab3-GAP p130) (Rab3- | | | |
| | GAP) | | | |
| P46934 | E3 ubiquitin-protein ligase NEDD4 (EC | 1319 | 149114 | 43.59 |
| | 2.3.2.26) (Cell proliferation-inducing gene | | | |
| | 53 protein) (HECT-type E3 ubiquitin | | | |
| | transferase NEDD4) (Neural precursor cell | | | |
| | expressed developmentally down-regulated | | | |
| | protein 4) (NEDD-4) | | | |
| P40937 | Replication factor C subunit 5 (Activator 1 | 340 | 38497 | 168.84 |
| | 36 kDa subunit) (A1 36 kDa subunit) | | | |
| | (Activator 1 subunit 5) (Replication factor C | | | |
| | 36 kDa subunit) (RF-C 36 kDa subunit) | | | |
| | (RFC36) | | | |
| Q9NQ66 | 1-phosphatidylinositol 4,5-bisphosphate | 1216 | 138567 | 47.63 |
| | phosphodiesterase beta-1 (EC 3.1.4.11) | | | |
| | (PLC-154) (Phosphoinositide phospholipase | | | |
| | C-beta-1) (Phospholipase C-I) (PLC-I) | | | |
| | (Phospholipase C-beta-1) (PLC-beta-1) | | | |
| Q5H9R7 | Serine/threonine-protein phosphatase 6 | 873 | 97669 | 67.58 |
| | regulatory subunit 3 (SAPS domain family | | | |
| | member 3) (Sporulation-induced transcript 4- | | | |
| | associated protein SAPL) | | | |
| Q9P2D3 | HEAT repeat-containing protein 5B | 2071 | 224302 | 30.32 |
| Q9NQX3 | Gephyrin [Includes: Molybdopterin | 736 | 79748 | 86.52 |
| | adenylyltransferase (MPT | | | |
| | adenylyltransferase) (EC 2.7.7.75) (Domain | | | |
| | G); Molybdopterin molybdenumtransferase | | | |
| | (MPT Mo-transferase) (EC 2.10.1.1) | | | |
| | (Domain E)] | | | |
| O75342 | Arachidonate 12-lipoxygenase, 12R-type | 701 | 80356 | 87.11 |
| | (12R-LOX) (12R-lipoxygenase) (EC | | | |
| | 1.13.11.-) (Epidermis-type lipoxygenase 12) | | | |
| P23919 | Thymidylate kinase (EC 2.7.4.9) (dTMP | 212 | 23819 | 293.88 |
| | kinase) | | | |
| Q02108 | Guanylate cyclase soluble subunit alpha-1 | 690 | 77452 | 91.67 |
| | (GCS-alpha-1) (EC 4.6.1.2) (Guanylate | | | |
| | cyclase soluble subunit alpha-3) (GCS-alpha- | | | |
| | 3) (Soluble guanylate cyclase large subunit) | | | |
| Q96RS6 | NudC domain-containing protein 1 (Chronic | 583 | 66756 | 109.35 |
| | myelogenous leukemia tumor antigen 66) | | | |
| | (Tumor antigen CML66) | | | |
| Q8N0X4 | Citramalyl-CoA lyase, mitochondrial (EC | 340 | 37359 | 195.40 |
| | 4.1.3.25) ((3S)-malyl-CoA thioesterase) (EC | | | |
| | 3.1.2.30) (Beta-methylmalate synthase) (EC | | | |
| | 2.3.3.-) (Citrate lyase subunit beta-like | | | |
| | protein) (Citrate lyase beta-like) (Malate | | | |
| | synthase) (EC 2.3.3.9) | | | |
| Q14005 | Pro-interleukin-16 [Cleaved into: | 1332 | 141752 | 51.50 |
| | Interleukin-16 (IL-16) (Lymphocyte | | | |
| | chemoattractant factor) (LCF)] | | | |
| Q7L5Y1 | Mitochondrial enolase superfamily member | 443 | 49786 | 146.63 |
| | 1 (EC 4.2.1.68) (Antisense RNA to | | | |
| | thymidylate synthase) (rTS) (L-fuconate | | | |
| | dehydratase) | | | |
| Q8TB24 | Ras and Rab interactor 3 (Ras | 985 | 107854 | 68.61 |
| | interaction/interference protein 3) | | | |
| Q8WW59 | SPRY domain-containing protein 4 | 207 | 23098 | 324.70 |
| Q96CW1 | AP-2 complex subunit mu (AP-2 mu chain) | 435 | 49655 | 151.04 |
| | (Adaptin-mu2) (Adaptor protein complex | | | |
| | AP-2 subunit mu) (Adaptor-related protein | | | |
| | complex 2 subunit mu) (Clathrin assembly | | | |
| | protein complex 2 mu medium chain) | | | |
| | (Clathrin coat assembly protein AP50) | | | |
| | (Clathrin coat-associated protein AP50) | | | |
| | (HA2 50 kDa subunit) (Plasma membrane | | | |
| | adaptor AP-2 50 kDa protein) | | | |
| P19404 | NADH dehydrogenase [ubiquinone] | 249 | 27392 | 277.45 |
| | flavoprotein 2, mitochondrial (NDUFV2) | | | |
| | (EC 7.1.1.2) (NADH-ubiquinone | | | |
| | oxidoreductase 24 kDa subunit) | | | |
| Q9Y305 | Acyl-coenzyme A thioesterase 9, | 439 | 49902 | 152.30 |
| | mitochondrial (Acyl-CoA thioesterase 9) | | | |
| | (EC 3.1.2.-) (Acyl-CoA thioester hydrolase | | | |
| | 9) | | | |
| O60341 | Lysine-specific histone demethylase 1A (EC | 852 | 92903 | 82.88 |
| | 1.14.99.66) (BRAF35-HDAC complex | | | |
| | protein BHC110) (Flavin-containing amine | | | |
| | oxidase domain-containing protein 2) | | | |
| | ([histone H3]-dimethyl-L-lysine(4) FAD- | | | |
| | dependent demethylase 1A) | | | |
| O60504 | Vinexin (SH3-containing adapter molecule | 671 | 75341 | 102.20 |
| | 1) (SCAM-1) (Sorbin and SH3 domain- | | | |
| | containing protein 3) | | | |
| P12694 | 2-oxoisovalerate dehydrogenase subunit | 445 | 50471 | 152.56 |
| | alpha, mitochondrial (EC 1.2.4.4) (Branched- | | | |
| | chain alpha-keto acid dehydrogenase E1 | | | |
| | component alpha chain) (BCKDE1A) | | | |
| | (BCKDH E1-alpha) | | | |
| Q15080 | Neutrophil cytosol factor 4 (NCF-4) | 339 | 39032 | 199.84 |
| | (Neutrophil NADPH oxidase factor 4) (SH3 | | | |
| | and PX domain-containing protein 4) (p40- | | | |
| | phox) (p40phox) | | | |
| P36776 | Lon protease homolog, mitochondrial (EC | 959 | 106489 | 74.19 |
| | 3.4.21.53) (LONHs) (Lon protease-like | | | |
| | protein) (LONP) (Mitochondrial ATP- | | | |
| | dependent protease Lon) (Serine protease 15) | | | |
| P21912 | Succinate dehydrogenase [ubiquinone] iron- | 280 | 31630 | 249.76 |
| | sulfur subunit, mitochondrial (EC 1.3.5.1) | | | |
| | (Iron-sulfur subunit of complex II) (Ip) | | | |
| | (Malate dehydrogenase [quinone] iron-sulfur | | | |
| | subunit) (EC 1.1.5.-) | | | |
| P43155 | Carnitine O-acetyltransferase (Carnitine | 626 | 70858 | 114.31 |
| | acetylase) (EC 2.3.1.137) (EC 2.3.1.7) | | | |
| | (Carnitine acetyltransferase) (CAT) (CrAT) | | | |
| Q9UGP4 | LIM domain-containing protein 1 | 676 | 72190 | 112.20 |
| Q9H336 | Cysteine-rich secretory protein LCCL | 500 | 56888 | 144.14 |
| | domain-containing 1 (CocoaCrisp) | | | |
| | (Cysteine-rich secretory protein 10) (CRISP- | | | |
| | 10) (LCCL domain-containing cysteine-rich | | | |
| | secretory protein 1) (Trypsin inhibitor Hl) | | | |
| Q9Y5X3 | Sorting nexin-5 | 404 | 46816 | 175.15 |
| P16885 | 1-phosphatidylinositol 4,5-bisphosphate | 1265 | 147870 | 55.45 |
| | phosphodiesterase gamma-2 (EC 3.1.4.11) | | | |
| | (Phosphoinositide phospholipase C-gamma- | | | |
| | 2) (Phospholipase C-IV) (PLC-IV) | | | |
| | (Phospholipase C-gamma-2) (PLC-gamma- | | | |
| | 2) | | | |
| Q9UI12 | V-type proton ATPase subunit H (V-ATPase | 483 | 55883 | 148.52 |
| | subunit H) (Nef-binding protein 1) (NBP1) | | | |
| | (Protein VMA13 homolog) (V-ATPase | | | |
| | 50/57 kDa subunits) (Vacuolar proton pump | | | |
| | subunit H) (Vacuolar proton pump subunit | | | |
| | SFD) | | | |
| O43837 | Isocitrate dehydrogenase [NAD] subunit | 385 | 42184 | 196.76 |
| | beta, mitochondrial (Isocitric dehydrogenase | | | |
| | subunit beta) (NAD(+)-specific ICDH | | | |
| | subunit beta) | | | |
| P32019 | Type II inositol 1,4,5-trisphosphate 5- | 993 | 112852 | 74.43 |
| | phosphatase (EC 3.1.3.36) (75 kDa inositol | | | |
| | polyphosphate-5-phosphatase) | | | |
| | (Phosphoinositide 5-phosphatase) (5PTase) | | | |
| Q16549 | Proprotein convertase subtilisin/kexin type 7 | 785 | 86247 | 97.39 |
| | (EC 3.4.21.-) (Lymphoma proprotein | | | |
| | convertase) (Prohormone convertase 7) | | | |
| | (Proprotein convertase 7) (PC7) (Proprotein | | | |
| | convertase 8) (PC8) (hPC8) | | | |
| | (Subtilisin/kexin-like protease PC7) | | | |
| Q13459 | Unconventional myosin-IXb | 2157 | 243401 | 34.51 |
| | (Unconventional myosin-9b) | | | |
| Q16740 | ATP-dependent Clp protease proteolytic | 277 | 30180 | 281.64 |
| | subunit, mitochondrial (EC 3.4.21.92) | | | |
| | (Endopeptidase Clp) | | | |
| Q99873 | Protein arginine N-methyltransferase 1 (EC | 371 | 42462 | 200.18 |
| | 2.1.1.319) (Histone-arginine N- | | | |
| | methyltransferase PRMT1) (Interferon | | | |
| | receptor 1-bound protein 4) | | | |
| Q5VWZ2 | Lysophospholipase-like protein 1 (EC | 237 | 26316 | 326.80 |
| | 3.1.2.22) | | | |
| O60242 | Adhesion G protein-coupled receptor B3 | 1522 | 171518 | 50.14 |
| | (Brain-specific angiogenesis inhibitor 3) | | | |
| Q96BW5 | N-acetyltaurine hydrolase (EC 3.1.-.-) | 349 | 39018 | 220.41 |
| | (Phosphotriesterase-related protein) | | | |
| O00273 | DNA fragmentation factor subunit alpha | 331 | 36522 | 238.21 |
| | (DNA fragmentation factor 45 kDa subunit) | | | |
| | (DFF-45) (Inhibitor of CAD) (ICAD) | | | |
| Q99747 | Gamma-soluble NSF attachment protein | 312 | 34746 | 253.27 |
| | (SNAP-gamma) (N-ethylmaleimide-sensitive | | | |
| | factor attachment protein gamma) | | | |
| Q05193 | Dynamin-1 (EC 3.6.5.5) (Dynamin) | 864 | 97408 | 90.34 |
| | (Dynamin I) | | | |
| Q4G0F5 | Vacuolar protein sorting-associated protein | 336 | 39155 | 224.75 |
| | 26B (Vesicle protein sorting 26B) | | | |
| P53582 | Methionine aminopeptidase 1 (MAP 1) | 386 | 43215 | 205.95 |
| | (MetAP 1) (EC 3.4.11.18) (Peptidase M 1) | | | |
| O75746 | Electrogenic aspartate/glutamate antiporter | 678 | 74762 | 121.72 |
| | SLC25A12, mitochondrial (Araceli | | | |
| | hiperlarga) (Aralar) (Aralar1) (Mitochondrial | | | |
| | aspartate glutamate carrier 1) (Solute carrier | | | |
| | family 25 member 12) | | | |
| Q9Y316 | Protein MEMO1 (C21orf19-like protein) | 297 | 33733 | 269.77 |
| | (Hepatitis C virus NS5A-transactivated | | | |
| | protein 7) (HCV NS5A-transactivated | | | |
| | protein 7) (Mediator of ErbB2-driven cell | | | |
| | motility 1) (Mediator of cell motility 1) | | | |
| | (Memo-1) | | | |
| Q9UBF9 | Myotilin (57 kDa cytoskeletal protein) | 498 | 55395 | 164.27 |
| | (Myofibrillar titin-like Ig domains protein) | | | |
| | (Titin immunoglobulin domain protein) | | | |
| O75746 | Electrogenic aspartate/glutamate antiporter | 678 | 74762 | 121.72 |
| | SLC25A12, mitochondrial (Araceli | | | |
| | hiperlarga) (Aralar) (Aralar1) (Mitochondrial | | | |
| | aspartate glutamate carrier 1) (Solute carrier | | | |
| | family 25 member 12) | | | |
| O43615 | Mitochondrial import inner membrane | 452 | 51356 | 177.19 |
| | translocase subunit TIM44 | | | |
| Q14108 | Lysosome membrane protein 2 (85 kDa | 478 | 54290 | 169.46 |
| | lysosomal membrane sialoglycoprotein) | | | |
| | (LGP85) (CD36 antigen-like 2) (Lysosome | | | |
| | membrane protein II) (LIMP II) (Scavenger | | | |
| | receptor class B member 2) (CD antigen | | | |
| | CD36) | | | |
| Q9Y450 | HBS1-like protein (EC 3.6.5.-) (ERFS) | 684 | 75473 | 121.90 |
| Q14BN4 | Sarcolemmal membrane-associated protein | 828 | 95198 | 96.64 |
| | (Sarcolemmal-associated protein) | | | |
| P11172 | Uridine 5'-monophosphate synthase (UMP | 480 | 52222 | 176.17 |
| | synthase) [Includes: Orotate | | | |
| | phosphoribosyltransferase (OPRT) | | | |
| | (OPRTase) (EC 2.4.2.10); Orotidine 5'- | | | |
| | phosphate decarboxylase (ODC) (OMPD) | | | |
| | (EC 4.1.1.23) (OMPdecase)] | | | |
| Q9Y334 | von Willebrand factor A domain-containing | 891 | 96060 | 95.77 |
| | protein 7 (Protein G7c) | | | |
| Q6UX06 | Olfactomedin-4 (OLM4) (Antiapoptotic | 510 | 57280 | 162.36 |
| | protein GW112) (G-CSF-stimulated clone 1 | | | |
| | protein) (hGC-1) (hOLfD) | | | |
| Q9NVG8 | TBC1 domain family member 13 | 400 | 46554 | 199.77 |
| Q8N3G9 | Transmembrane protein 130 | 435 | 48329 | 194.50 |
| P28289 | Tropomodulin-1 (Erythrocyte tropomodulin) | 359 | 40569 | 231.70 |
| | (E-Tmod) | | | |
| Q9UKM9 | RNA-binding protein Raly (Autoantigen | 306 | 32463 | 289.56 |
| | p542) (Heterogeneous nuclear | | | |
| | ribonucleoprotein C-like 2) (hnRNP core | | | |
| | protein C-like 2) (hnRNP associated with | | | |
| | lethal yellow protein homolog) | | | |
| Q5T6V5 | Queuosine 5'-phosphate N- | 341 | 39029 | 240.85 |
| | glycosylase/hydrolase (EC 3.2.2.-) (Q- | | | |
| | nucleotide N-glycosylase 1) (Queuine | | | |
| | salvage protein QNG1) (Queuosine- | | | |
| | nucleotide N-glycosylase/hydrolase) | | | |
| Q9UDY4 | DnaJ homolog subfamily B member 4 (Heat | 337 | 37807 | 251.28 |
| | shock 40 kDa protein 1 homolog) (HSP40 | | | |
| | homolog) (Heat shock protein 40 homolog) | | | |
| | (Human liver DnaJ-like protein) | | | |
| O75829 | Leukocyte cell-derived chemotaxin 1 | 334 | 37102 | 256.05 |
| | (Chondromodulin) [Cleaved into: | | | |
| | Chondrosurfactant protein (CH-SP); | | | |
| | Chondromodulin-1 (Chondromodulin-I) | | | |
| | (ChM-I)] | | | |
| P22676 | Calretinin (CR) (29 kDa calbindin) | 271 | 31540 | 304.38 |
| Q6P996 | Pyridoxal-dependent decarboxylase domain- | 788 | 86707 | 110.72 |
| | containing protein 1 (EC 4.1.1.-) | | | |
| Q99700 | Ataxin-2 (Spinocerebellar ataxia type 2 | 1313 | 140283 | 68.43 |
| | protein) (Trinucleotide repeat-containing | | | |
| | gene 13 protein) | | | |
| P22676 | Calretinin (CR) (29 kDa calbindin) | 271 | 31540 | 304.38 |
| Q6PK18 | 2-oxoglutarate and iron-dependent | 319 | 35646 | 272.12 |
| | oxygenase domain-containing protein 3 (EC | | | |
| | 1.14.11.-) | | | |
| Q8NBJ5 | Procollagen galactosyltransferase 1 (EC | 622 | 71636 | 135.41 |
| | 2.4.1.50) (Collagen beta(1- | | | |
| | O)galactosyltransferase 1) (ColGalT 1) | | | |
| | (Glycosyltransferase 25 family member 1) | | | |
| | (Hydroxylysine galactosyltransferase 1) | | | |
| Q9Y4D1 | Disheveled-associated activator of | 1078 | 123473 | 78.56 |
| | morphogenesis 1 | | | |
| Q6P2E9 | Enhancer of mRNA-decapping protein 4 | 1401 | 151661 | 63.96 |
| | (Autoantigen Ge-1) (Autoantigen RCD-8) | | | |
| | (Human enhancer of decapping large | | | |
| | subunit) (Hedls) | | | |
| Q9NS84 | Carbohydrate sulfotransferase 7 (EC 2.8.2.-) | 486 | 54266 | 180.59 |
| | (EC 2.8.2.17) (Chondroitin 6- | | | |
| | sulfotransferase 2) (C6ST-2) (Galactose/N- | | | |
| | acetylglucosamine/N-acetylglucosamine 6- | | | |
| | O-sulfotransferase 5) (GST-5) (N- | | | |
| | acetylglucosamine 6-O-sulfotransferase 4) | | | |
| | (GlcNAc6ST-4) (Gn6st-4) | | | |
| Q5T1M5 | FK506-binding protein 15 (FKBP-15) (133 | 1219 | 133630 | 73.34 |
| | kDa FK506-binding protein) (133 kDa | | | |
| | FKBP) (FKBP-133) (WASP- and FKBP-like | | | |
| | protein) (WAFL) | | | |
| Q5M775 | Cytospin-B (Nuclear structure protein 5) | 1068 | 118585 | 82.64 |
| | (NSP5) (Sperm antigen HCMOGT-1) | | | |
| | (Sperm antigen with calponin homology and | | | |
| | coiled-coil domains 1) | | | |
| O43592 | Exportin-T (Exportin(tRNA)) (tRNA | 962 | 109964 | 89.12 |
| | exportin) | | | |
| Q5T1M5 | FK506-binding protein 15 (FKBP-15) (133 | 1219 | 133630 | 73.34 |
| | kDa FK506-binding protein) (133 kDa | | | |
| | FKBP) (FKBP-133) (WASP- and FKBP-like | | | |
| | protein) (WAFL) | | | |
| P25686 | DnaJ homolog subfamily B member 2 (Heat | 324 | 35580 | 278.25 |
| | shock 40 kDa protein 3) (Heat shock protein | | | |
| | J1) (HSJ-1) | | | |
| Q9BW91 | ADP-ribose pyrophosphatase, mitochondrial | 350 | 39125 | 253.04 |
| | (EC 3.6.1.13) (ADP-ribose diphosphatase) | | | |
| | (ADP-ribose phosphohydrolase) (Adenosine | | | |
| | diphosphoribose pyrophosphatase) (ADPR- | | | |
| | PPase) (Nucleoside diphosphate-linked | | | |
| | moiety X motif 9) (Nudix motif 9) | | | |
| P62166 | Neuronal calcium sensor 1 (NCS-1) | 190 | 21879 | 457.06 |
| | (Frequenin homolog) (Frequenin-like | | | |
| | protein) (Frequenin-like ubiquitous protein) | | | |

### High Reproducibility

Reproducibility is a critical requirement for clinical diagnostics, as it ensures precision and reliability in assay results. With clinical applications as the focus, Applicants systematically evaluated the reproducibility of the Complete360® assay using two complementary approaches:

Applicants evaluate the detection and quantification of 36 proteins across 12 replicates. These proteins span a wide dynamic range of documented plasma concentrations, from 19 ng/L to 25 mg/L, covering over 106 orders of magnitude (Table 1). By including proteins at varying concentrations in plasma, reproducibility across a wide dynamic range is carefully assessed, highlighting the robustness and consistency of the Complete360® platform. Remarkably, the results demonstrated high reproducibility, with an average coefficient of variation (CV) in quantification of only 3.92% ranging from 1.4% to 7.0% across all proteins on the panel (Table 1). Representative raw spectra of key proteins illustrate this exceptional reproducibility. These findings demonstrate that the Complete360® assay is highly reproducible, meeting the stringent requirements necessary for clinical applications.

To assess a broader range of proteins, Applicants conducted Complete360® assays targeting 9,977 proteins across five replicates (FIG. 8, Table 5). The median CV for the entire panel is 11.97%. When focusing on proteins with CVs below 25%, 7,833 proteins are consistently detected across all replicates, with a median CV of 8.73%. Notably, 4,361 proteins exhibited CVs below 10%, with a median CV of 4.77%. This subset of highly reproducible proteins demonstrates significant potential for direct translation into clinical applications once a clinical relevance to a disease is validated.

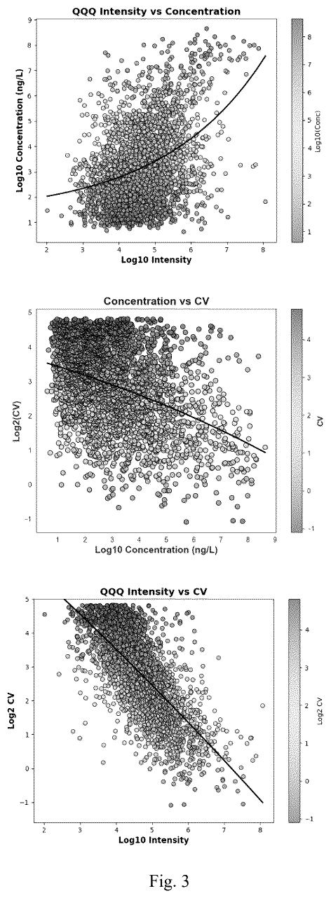

It is noteworthy that the reproducibility of protein abundance measurements and signal intensity detected by the Complete360® platform demonstrates a strong correlation (FIG. 3). As the biological concentration of proteins in plasma increases, the reproducibility of detection improves accordingly. Moreover, the Complete360® platform reliably quantifies protein targets across a dynamic range exceeding eight orders of magnitude. The correlation between QqQ intensity and reproducibility is remarkably high, with an R² value of 0.51. It is also important to highlight that only about 4,500 proteins have documented blood concentration data to date²⁵. Interestingly, most of the proteins included in the Complete360® full panel are not yet documented for their blood concentration levels. Future efforts will focus on systematically establishing these concentration profiles to further enhance the clinical and diagnostic utility of the current platform, including achieving reliable absolute quantifications.

Next, Applicants compare the reproducibility and detectability of Complete360® to conventional DIA-based proteomic profiling. Using timsTOF HT to analyze the same sample, three technical replicates are conducted, resulting in the identification of 7,697 proteins using DIA-MS. (Table 6). Among these, 3,944 proteins are consistently detected across all three replicates, 2,737 proteins are observed in two out of three replicates, and 1,016 proteins were identified in only one replicate. For proteins detected in two or more replicates, the median CV for quantification was calculated to be 15.23%, indicating a high-quality profiling assay. These results demonstrate that Complete360®, with its optimized parameters, provides substantially enhanced reproducibility and quantification consistency compared to conventional DIA-based approaches.

The findings demonstrated the reproducibility of the Complete360® platform across both small- and large-scale protein panels, covering a wide dynamic range of protein concentrations. The ability to consistently achieve results with CVs well within acceptable thresholds highlights Complete360® as a reliable and practical solution for clinical applications. Its enhanced reproducibility and sensitivity, offering improved reliability and confidence in detection and quantification plasma proteins, making Complete360® a valuable tool for mass spectrometry-based proteomics, particularly in clinical diagnostics where precision and consistency are essential.

### Disease-Associated Molecular Changes Revealed by Complete360®

Applicants conduct Complete360® assays on 102 plasma samples obtained from biobanks, representing patients diagnosed with a range of diseases, including breast cancer, colorectal cancer, lung cancer, ovarian cancer, pancreatic cancer, prostate cancer, ulcerative colitis, and Alzheimer's disease. The results reveal clear disease clustering within their respective categories (FIG. 4), which distinctive biomarker patterns for each condition are further analyzed to identify.

Using the Complete360® platform, Applicants identify a range of potential target proteins specific to each disease, including 386 for breast cancer, 288 for colorectal cancer, 226 for lung cancer, 209 for ovarian cancer, 195 for pancreatic cancer, 331 for prostate cancer, 280 for ulcerative colitis, and 407 for Alzheimer's disease (Table 7). Notably, a significant proportion of these proteins have already been documented in association with the respective diseases. Specifically, 319, 199, 172, 121, 112, 192, 64, and 246 proteins discovered above were previously identified for these conditions confirming the feasibility of findings. In addition, the proportion of previously reported proteins in current validated biomarker panels varied, ranging from 23% for ulcerative colitis to 83% for breast cancer, which likely reflects the popularity of research on each disease.

These findings not only confirm the relevance of established protein targets identified by Complete360® but also highlight the platform's potential in uncovering novel disease-associated proteins. For instance, 17%, 31%, 24%, 42%, 43%, 42%, 77%, and 40% of the protein targets identified for each disease had never been reported before, underscoring Complete360® 's ability to contribute to disease-associated target discovery.

Breast Cancer. In breast cancer, several key proteins linked to tumor progression and metastasis were identified. Notable proteins include eIF4E type 2, whose overexpression is associated with more aggressive breast cancer forms, and vinculin, involved in cell migration and metastasis^{26, 27}. Serpin B6, regulating protease activity, has been shown to influence metastasis and progression²⁸. Ficolin-2, an immune system protein, plays a role in immune surveillance, and Large ribosomal subunit protein eL22, frequently deregulated in cancers, suggests its role in tumor biology^{29, 30}. Lastly, Serine/arginine-rich splicing factor 7 (SRSF7), which regulates RNA splicing, exhibits altered patterns in breast cancer, contributing to tumorigenesis³¹.

Colorectal Cancer. For colorectal cancer, several biomarkers have been well-documented in the literature. These include Alanine aminotransferase 2 (ALT2), whose elevated expression correlates with liver metastases, and C-C motif chemokine 19 (MIP-3-beta), associated with tumor progression^{32, 33}. RhoE (Rnd3), which regulates cell cycle and epithelial-mesenchymal transition (EMT), and Ubiquitin-conjugating enzyme E2 B (hHR6B), critical for protein degradation, are both implicated in colorectal cancer metastasis^{34, 35}. Additionally, Cytochrome P450 26C1 (CYP26C1) and CCN family member 2 (CTGF) are involved in cancer metabolism and progression, respectively^{36, 37}.

Lung Cancer. In lung cancer, proteins like hPEAR1, involved in endothelial function, and MASP-1, linked to immune response, were identified as biomarkers^{38, 39}. BRSK2, regulating the cell cycle, and THBS7A, contributing to lung cancer angiogenesis and metastasis, were also notable hits^{40, 41} Other relevant proteins such as Secernin-1 and TANC2 suggest broader impacts on apoptosis and immune modulation, which are critical for lung cancer progression^{42, 43}.

Ovarian Cancer. Key biomarkers for ovarian cancer identified through Complete360® include RhoGAP23, linked to cell migration, and Tentonin 3, which plays a role in cellular adhesion^{44, 45}. FAF-Y, a deubiquitinating enzyme, and CIP29, involved in RNA processing, were also highlighted^{46, 47}. Additional proteins like DGK-alpha, TRAF1, and TBX18 represent promising candidates for further exploration in diagnosis and therapy⁴⁸⁻⁵⁰.

Pancreatic Cancer. For pancreatic cancer, known biomarkers such as PKN gamma, involved in actin dynamics, and SGA-72M, linked to vesicle trafficking and metastasis, were identified^{51, 52}. USP35, regulating protein degradation, and Beta-catenin, a key player in Wnt signaling, emerged as significant pancreatic cancer biomarkers^{53, 54}. Additionally, proteins like ITI-HC4, KIF2B, and NPRL3 play roles in inflammation, cell proliferation, and tumor metabolism, underscoring their roles in pancreatic cancer progression have been previously reported^{55, 56}.

Prostate Cancer. Prostate cancer biomarkers identified through Complete360® include Beta-2-microglobulin, associated with advanced disease stages, and CYP1B1, involved in carcinogen metabolism^{57, 58} LPL, dysregulated in prostate cancer, and CC3, linked to inflammatory responses, were also highlighted^{59, 60}. Other key proteins, such as SEC63 homolog and srGAP2, are associated with tumor progression and metastasis^{61, 62}.

Ulcerative Colitis (UC). In UC, several biomarkers such as GDF-8 (Myostatin) and Alpha-2-macroglobulin, involved in systemic inflammation, were identified^{63, 64}. DeSI-1, a protein regulating immune pathways, and Sodium/potassium/calcium exchanger 2, essential for epithelial ion transport, are also implicated^{65, 66}. Dysregulation of Cytoskeleton-associated protein 2 suggests a role in impaired epithelial barrier function, a hallmark of UC pathology⁶⁷.

Alzheimer's Disease. For Alzheimer's disease, biomarkers like LRRTM4, linked to synaptic function, and NADH dehydrogenase [ubiquinone] 1 alpha subcomplex subunit 5, associated with mitochondrial dysfunction, were identified^{68, 69}. CD166 antigen (ALCAM), Serpin B6, and DDX39B, involved in neuroinflammation and protein aggregation, were also highlighted⁷⁰⁻⁷². Additionally, Syntaxin-10 and FHR-2, implicated in vesicle trafficking and immune modulation, are crucial for understanding AD pathology^{73, 74}.

### Simultaneous Quantification of Plasma Proteins, Metabolites, and Lipids for Enhanced Diagnostic Precision

To achieve comprehensive diagnostics, the Complete360® platform has been integrated with metabolomics and lipidomics analysis, enabling the simultaneous detection and quantification of proteins, metabolites, and lipids from the same biological sample through the same platform. This streamlined multi-omics approach maximizes sample utilization, enhances diagnostic accuracy while maintaining cost efficiency. By consolidating all assays onto a unified platform, current method facilitates seamless clinical implementation, supporting broader adoption in clinical and translational research.

Current Complete360®-MyMeta assay employs a targeted method capable of detecting 762 metabolites and 1,395 lipids, with systematic optimization across multiple refinement cycles to enhance sensitivity and reproducibility. The metabolomics assays cover both polar metabolites and lipids, with data undergoing separate median normalization for metabolites (Table 8) and lipids (Table 9).

For each disease, current integrated approach has yielded highly informative results, revealing matched metabolic and proteomic signatures (Table 10). Notably, pathway analysis shows strong concordance between proteomic and metabolomic data, with an average of 69% of the top 10 pathways concerning metabolomics identified from each omics perspective overlapping. This alignment underscores the robustness and biological relevance of the integrated Complete360® platform in disease characterization.

To fully leverage the capabilities of the multi-omics Complete360® platform, Applicants integrate proteins, metabolites, and lipids as key features and performed a t-test to compare each disease against all other conditions. Molecules are ranked in ascending order based on p-values, and the top 1,000 features are consistently selected for model construction. ROC curves are then generated using only the top 1,000 proteins for each disease and compared to ROC curves generated when the top 1,000 features—comprising proteins, polar metabolites, and lipids—were collectively incorporated (FIG. 4B). To ensure consistency, the total feature count is always maintained at 1,000, allowing the model to determine the optimal composition of proteins, metabolites, and lipids for each disease-specific diagnostic panel. On average, 794 proteins are selected, while the mean feature count for metabolites and lipids is 90 and 117, respectively (Table 11). Notably, an increase in AUC values is observed for most diseases when multi-omics features were incorporated into the diagnostic model, underscoring the advantage of integrating multiple molecular layers (FIG. 4B).

TABLE 11
Feature Selection Count by Category for Each Disease Diagnosis

| Sample | Lipid | Metabolite | Peptides | Sum |
|---|---|---|---|---|
| AD | 90 | 148 | 762 | 1000 |
| BC | 109 | 9 | 882 | 1000 |
| CRC | 50 | 75 | 875 | 1000 |
| PROSC | 150 | 96 | 754 | 1000 |
| Average | 100 | 82 | 818 | 1000 |
| Stdev | 42 | 58 | 70 | 0 |

This enhancement highlights the unique value of the Complete360® platform, which enables the simultaneous and cost-effective analysis of multi-omics analytes on a single instrument, improving both diagnostic accuracy and efficiency. Applicants acknowledge that the current study is based on a limited sample size, and while the observed ROC curves provide valuable insights, they may not fully capture the diagnostic potential of Applicants' approach. Future studies with larger sample cohorts and deeper data analysis approaches will be conducted to further validate these findings, as this work primarily serves as a proof-of-concept demonstration of the Complete360® platform.

### Plasma Proteome Variation and Its Genetic Determinants Revealed by Complete360®

Using Complete360® methods, Applicants conduct an ultra-deep plasma proteomics analysis to investigate the correlation between plasma protein levels and human age, gender, and BMI. The findings align closely with those reported by Mann et al., demonstrating that a significant proportion of the plasma proteome varies systematically with these demographic and physiological factors⁷⁵. Notably, age-, gender- or BMI-associated shifts in proteins have been identified involved in inflammation, extracellular matrix remodeling, lipid metabolism, and coagulation cascades, reflecting the dynamic changes in systemic physiology (FIG. 5).

Complete360®'s high-sensitivity protein detection enabled the identification of BMI-associated proteomic signatures, uncovering key proteins involved in metabolic regulation, inflammatory response, and lipid transport (FIG. 5). Notably, proteins such as TRIB3 (a regulator of obesity and insulin resistance), INHBE (a determinant of fat distribution), and ERBB4 (which modulates brain-regulated energy expenditure) exhibited distinct expression profiles across BMI categories. Among these, LEP (leptin) emerges as a particularly significant contributor to BMI, reinforcing its well-documented role in weight regulation⁷⁶. These findings reveal a robust molecular signature of metabolic health, providing a valuable framework for biomarker discovery and disease risk stratification. The strong correlation between these plasma proteins and BMI underscores the predictive power of Complete360® in distinguishing metabolic states. This highlights the potential of plasma proteomics not only as a biological clock for metabolic health but also as an innovative tool for early disease detection and personalized health monitoring.

Furthermore, Complete360® facilitates genetic-proteomic association studies (pQTL analysis) to determine the genetic influences on plasma protein levels. The initial findings suggest that genetic variants contribute significantly to the observed protein-level variance, with some proteins showing strong cis- and trans-regulatory effects. The integration of Complete360™ with genome-wide association studies (GWAS) is expected to further uncover causal relationships between genetic factors, proteomic alterations, and disease predisposition.

With its ability to quantify thousands of plasma proteins at high specificity, capture proteomic variability with minimal technical noise, and support predictive modeling of age and BMI, Complete360® is at the forefront of precision medicine and multi-omics biomarker research. These insights will be instrumental in improving disease risk assessment, enhancing therapeutic targeting, and advancing the understanding of human health at the molecular level.

### Complete360® Assay for Dried Blood Spot Samples

Dried blood spot (DBS) sampling is a widely adopted method for at-home sample collection due to its convenience, ease of storage, and cost-effective transportation via standard mailing services. However, proteomics assays face substantial challenges when applied to DBS samples. Affinity-based proteomics methods often suffer from loss of protein epitope integrity and higher-order structural degradation during prolonged room-temperature storage. Similarly, mass spectrometry-based approaches are hindered by an overwhelming release of peptides from red blood cells and other cellular components, compromising the depth and specificity of plasma proteome analysis derived from whole blood DBS samples. The Complete360®. platform overcomes these limitations for DBS analysis. Unlike affinity-based methods that depend on intact epitopes, Complete360® utilizes a proprietary approach optimized for detecting targets that are inherently resistant to proteolysis and chemical modifications. These targets have been carefully selected and refined to enhance assay performance. Proteins released into the sample due to prolonged storage at room temperature do not interfere with the detection of desired targets, allowing the assay to maintain high sensitivity and specificity.

To assess the assay's performance under varied storage conditions, DBS samples are collected and stored at ambient temperature for 1 to 12 days in standard mailing envelopes, simulating routine transport scenarios (FIG. 6A). The samples are processed using Complete360® platform (Materials and Methods, FIG. 7) and analyzed for proteins and small molecules using the Complete360® pipeline.

Through Complete360®, over 10,000 proteins from DBS samples have been systematically analyzed. Approximately 2-3% of proteins exhibit consistent temporal changes, with 340 proteins showing a steady increase and 279 proteins showing a continuous decrease over storage periods of 1, 3, 5, 7, and 9 days. Additionally, 46.96% of proteins maintain a coefficient of variation (CV) below 25% across all DBS samples collected from the same individual, demonstrating the pipeline's stability and resistance to long-term exposure to air and room temperature (Table 12). Notably, plasma protein profiles from DBS samples closely match those from conventionally collected plasma samples from the same individual (FIG. 6B, 6C), supporting the reliability of Complete360® for proteomic analysis of DBS samples. However, further studies are needed to evaluate the diagnostic performance of using diseased DBS samples, as this was beyond the scope of the current study.

Based on these data, a normalization reference dataset encompassing all Complete360®-detected proteins over a 12-day period from the same individual has been developed (Table 12). This dataset enables future analyses to account for time-dependent variations in protein abundance, enhancing the accuracy of disease biomarker quantification from DBS samples. By integrating the mailing timestamp of DBS samples, this approach allows for precise adjustments to compensate for protein changes occurring during shipment and storage to best reflect the original clinical status of the patient when the DBS sample was collected.

Furthermore, a discovery-mode analysis using the timsTOF HT platform identifies 5,781 proteins from DBS samples, significantly expanding the depth of coverage compared to conventional plasma proteomics (FIG. 9). Notably, 133 proteins are uniquely detected in DBS samples and have not been previously observed in freshly frozen plasma and is outside of the CompleteBank database of 17,328 plasma proteins.

DBS is an inherently robust sample type for metabolomics and lipid assays. Using Complete360®, a comprehensive set of 1,395 metabolites and 762 lipids from DBS samples has been identified, demonstrating the feasibility of high-throughput multi-omics analysis. Notably, the levels of these small molecules remain remarkably stable across multiple days (Table 13), underscoring the reliability of DBS for longitudinal studies. These findings highlight the potential of DBS-based Complete360® analysis for clinical applications in disease detection and monitoring, with its utility strongly supported by the stability of the detected analytes in this dataset.

These findings underscore the exceptional versatility and robustness of the Complete360® platform in accommodating diverse sample collection and storage conditions, including long-distance transport and room-temperature storage of DBS samples. This capability has the potential to redefine clinical proteomics by enabling comprehensive multi-omics analyses from easily accessible and transportable biological specimens. The ability to derive deep proteomic and metabolic insights from DBS samples opens new avenues for remote patient monitoring, large-scale epidemiological studies, and global healthcare applications, ensuring the generation of high-quality multi-omics data regardless of logistical constraints.

### Discussion

Complete360® is a highly targeted and comprehensive detection platform, capable of quantifying close to 13,000 molecules from blood samples, delivers unmatched sensitivity and reproducibility, exceeding the capabilities of traditional profiling methods commonly used in academic and clinical settings. Underpinned by the comprehensive CompleteBank databases and CompletePeaking algorithms, Complete360® establishes itself as a potential transformative tool for basic research and clinical diagnostics, offering advancements in biomarker discovery, disease pathway analysis, and personalized medicine. It is designed to bridge the gap between multi-omics research and real-world clinical applications, enabling newly identified molecular changes to be seamlessly translated into clinical use on the same platform.

Proteomics assays generally follow two main approaches: mass spectrometry-based methods and affinity-based detection techniques. Mass spectrometry relies on advancements in instrumentation, sample preparation, and data analysis algorithms, while affinity-based methods employ antibodies or aptamers to facilitate assays such as ELISA or its variations, like proximity extension assays (PEA). Although affinity-based methods have been widely applied to clinical specimens, their limitations are apparent. They rely heavily on the quality of the binding reagents, which can lead to inconsistencies due to variations in the manufacturing of antibodies or aptamers⁷⁷⁻⁷⁹. Even with high-quality binding reagents, the detection may be hindered by the limited accessibility of epitopes; many proteins in blood are modified by different protein modifications or form complexes by binding to other molecules, obscuring their binding sites⁸⁰. Furthermore, many proteins that serve as valuable biomarkers and are involved in rapid physiological responses have relatively short half-lives therefore hindering their detection by affinity-based methods^{81, 82}. For example, insulin and glucagon, both critical for glucose regulation, have half-lives of about 5 to 10 minutes, while cytokines like interleukin-6 can range from minutes to a few hours. Although these short-lived proteins are essential disease biomarkers, accurately detecting them using affinity-based methods is challenging. This is due to epitope masking through binding to other proteins, or rapid epitope damage and degradation caused by protease digestion. These factors often result in compressed fold-change data in affinity-based methods, making it difficult to differentiate between disease and healthy individuals. As a result, the sensitivity and specificity required for effective diagnostics are significantly compromised.

Mass spectrometry-based proteomics offers superior specificity and resolution, making it well-suited for distinguishing disease from control samples. However, these methods also face challenges related to sensitivity and reproducibility. Most proteomics assays employ profiling techniques using orbitrap or time-of-flight mass analyzers, and these platforms often fall short of the reproducibility standards required for clinical applications, where a coefficient of variation (CV) below 10% is essential. While triple quadrupole mass spectrometers are widely used in clinical laboratories to detect disease-associated small molecules, they require extensive optimization of detection parameters, including sample preparation strategies. Despite efforts to establish standardized detection protocols using synthetic peptides, these parameters often remain theoretical and may not fully account for the variability of real-world clinical samples.

Given these challenges, there is an urgent need for a robust and reproducible proteomics platform capable of detecting a broad spectrum of clinically relevant proteins and metabolites from blood samples with high accuracy and reliability. This is the driving force behind the development of Complete360®. The platform is designed to provide a finely tuned, clinically viable system for comprehensive proteomic and metabolic analysis in blood, ensuring the reproducibility and sensitivity required for clinical applications. Through years of refinement, Applicants have optimized sample preparation workflows, established precise detection parameters, and developed a sophisticated data analysis pipeline. Validated through the analysis of a good amount of body fluid samples, Complete360® represents a major advancement in proteomics research and its translation into clinical practice. Looking ahead, Applicants aim to extend the application of Complete360® beyond basic research to direct clinical diagnostics. The goal is to implement this platform across multiple countries, facilitating improved disease detection and better patient outcomes. By bridging the gap between proteomics research and clinical application, Complete360® has the potential to redefine the future of precision medicine.

### III. Materials and Methods

### 1. Chemicals and Reagents

For blood proteomics: Water Optima® LS/MS grade; Acetonitrile Optima LC/MS grade; Methanol LC/MS grade; Ammonium Bicarbonate (ABC) IM; Tris buffered saline (Sigma), Formic Acid 98%-100%; Sodium dodecyl sulfate (SDS); Tris-(2-Carboxyethyl) phosphine-HCl (TCEP); 2-Chloroacetamide (CAA) ≥98%; Triethylammonium bicarbonate (TEAB) 1.0 M; Triethylamine; Phosphoric Acid; Promega sequencing grade Trypsin; Whatman 903™ blood collection kit, Minute™ albumin depletion kit. For small molecules: Ammonium formate, Ammonium acetate, Ammonium hydroxide solution: Sigma-Aldrich; Methanol (LC), water (LC/MS Grade), acetonitrile, and 2-propanol (LC/MS grade, LiChrosolv): Fisher Sci.

### 2. Patients and Samples

Plasma samples used in this study were obtained from BioIVT (Westbury, NY, USA), along with comprehensive clinical information (Table 14). All samples were collected in accordance with institutional ethical guidelines and were de-identified to ensure patient confidentiality. Plasma was collected using purple-top tubes containing EDTA as an anticoagulant. Upon collection, samples were processed promptly by centrifugation to separate plasma, aliquoted, and stored at −80° C. until further use to minimize freeze-thaw cycles and maintain proteomic and metabolic stability.

### 3. Plasma Sample Preparation and Analysis Methods

Plasma samples were processed using the Complete360®-MyProt pipeline, incorporating Applicants' Chemical-Biological Plasma Protein Preparation procedure. This workflow starts from two key steps to remove high abundance proteins and collect clinically and biologically more meaningful low-abundance proteins:

Chemical Procedure: Major plasma proteins were precipitated using a set of in-house-prepared protein removal reagents.
Biological Procedure: The remaining high-abundance and median-abundance plasma proteins were depleted using a proprietary antibody-conjugated resin, targeting a combination of proteins that are most frequently detected by mass spectrometry and reported in the database of peptide atlas. Such depletion procedure has been observed to be more reproducible for protein quantifications compared to that of nanoparticle-based plasma low-abundance protein enrichment methods (data not shown). Protein depletion methods for other body fluid can be established the same way. The depletion resin is tested for durability, demonstrating consistent performance for over 200 uses with optimized buffers and procedures (data not shown) to ensure an ultra-low cost for plasma protein extraction.

After the removal of high- and median-abundance proteins through this chemical-biological procedure, the plasma proteins remaining in the supernatant were processed into peptides using the Complete360® sample digestion kits and reagents. Briefly, plasma proteins were denatured using SDS and digested with an optimized trypsin digestion protocol. Following digestion, the resulting peptide samples were fractionated using an offline HPLC system operating in both low-pH and high-pH modes. This dual-mode approach ensures highly reproducible chromatographic profiles. The procedures were extensively optimized for human plasma samples, with key metrics such as protein identification, detected abundance, and mis-cleavage rates carefully monitored to ensure reproducibility and sensitivity of mass spectrometry analysis.

For proteomics analysis using dried blood spot (DBS) samples, three 12 mm disks were pooled and incubated in Tris-buffered saline (TBS) containing 0.05% NP-40 at 37° C. for 30 minutes with agitation. The supernatant was then combined with an equal volume of the Minute™ Albumin Depletion Kit reagent to remove albumin and hemoglobin; this depletion step was repeated twice. The resulting precipitate was solubilized in TBS and subjected to digestion using Complete360® sample digestion kits and reagents as described above.

Peptide digests were then subjected to basic reversed-phase chromatography using an Agilent 1260 liquid chromatography system, following the methodology outlined reported previously⁸³. Separation was performed on an in-house packed C18 column employing a gradient of acetonitrile in 10 mM triethylammonium bicarbonate (TEAB). The gradient conditions were as follows: 5% to 28% solvent B over 75 minutes, increased to 42% over the next 8 minutes, and then to 98% over the subsequent 3 minutes, at a flow rate of 1 mL/min. Fractions were collected every minute and were then concentrated to dryness using a SpeedVac equipped with a chilled vacuum trap. The dried peptides were stored at −80° C. until further analysis.

DIA-MS Analysis: Mass spectrometric discovery analyses were conducted using a timsTOF HT mass spectrometer coupled to a nanoElute® 2 liquid chromatography system via a CaptiveSpray™ ion source, configured in a two-column setup comprising a 5 mm Thermo trap cartridge and a PepSep Max Ten series analytical column (10 cm×150 μm i.d., 1.5 μm particle size).

DDA-PASEF Analysis: To assess the quality of trypsin digestion, including the evaluation of missed cleavages and potential artifacts, data-dependent acquisition parallel accumulation-serial fragmentation (DDA-PASEF) analyses were performed. This approach facilitated the identification of peptides and proteins, ensuring the integrity of the digestion process.

DIA-PASEF Analysis: For comprehensive proteomic profiling, data-independent acquisition PASEF (DIA-PASEF) analyses were executed. The acquisition method was optimized to minimize missing data and to cover ion mobility ranges with high-density precursor sampling. The method consisted of eight cycles, each comprising 29 ion mobility (IM) windows. An initial MS1 scan was followed by eight DIA-PASEF cycles, covering an m/z range of 375-1100 and an inverse reduced mobility (1/Ko) range of 0.65-1.45 V·s/cm². The resulting DIA-MS data were processed using DIA-NN (version 1.8.2) employing a predicted human protein spectral library containing 20,480 entries. Both DDA- and DIA-PASEF datasets were analyzed using DIA-NN and FragPipe software to ensure comprehensive identification and quantification of peptides and proteins^{84, 85}.

Complete360®-MyProt Analysis: Targeted detection and quantification for plasma proteins was performed on an Agilent 6495 QqQ Mass Spectrometer using dynamic Selected Reaction Monitoring (dSRM) with an Agilent Jet Stream ion source. Chromatographic separation was achieved using an in-house packed C18 column (1.7 μm, 2.1 mm×30 mm). The gradient of solvent B (acetonitrile with 0.1% formic acid) was programmed as follows: 12% to 42% over 5.4 minutes, followed by an increase to 98% in the next minute, at a flow rate of 150 μL/min. A set of targeted assays were created through CompleteBank-Discovered and CompleteBank-Validated process to therefore detect and quantify over 10,000 plasma proteins in the same assay.

### 4. Sample Preparation and Analysis Methods for Small Molecules

Plasma samples were processed using the Complete360®-MyMeta pipeline using a modified MTBE/MeOH/H2O extraction protocol to isolate lipids and polar metabolites from 40 μl plasma sample. First, 300 L of cold methanol was added to the plasma aliquot and vortexed for 10 seconds. Following this, 1 mL of methyl tert-butyl ether (MTBE) was added, and the mixture was vortexed again for 10 seconds before being incubated on a shaker at room temperature for 60 minutes. After incubation, 250 μL of water was added to induce phase separation, followed by a 10-minute incubation at room temperature with occasional vortexing. The samples were then centrifuged at 15,000 g for 10 minutes to separate the phases. The upper organic lipid phase (approximately 900 μL) was collected into a clean 2 mL glass vial, while the lower aqueous metabolite phase (320-350 μL) was also collected. The organic phase was dried under nitrogen gas for 20-30 minutes and reconstituted in 300 μL of 1-butanol/methanol (1:1) containing 10 mM ammonium formate. The aqueous phase was dried under nitrogen and reconstituted in 150 μL of 50% acetonitrile (ACN).

Complete360®-MyMeta Analysis: For MyMeta analysis, metabolites were separated on an Agilent 1290 LC system using two HILIC-LC methods. The first method, HILIC-01, employed an Acquity BEH-Amide column (1.7 μm, 2.1×150 mm) at a column temperature of 40° C. and an autosampler temperature of 8° C. The injection volume was 5 μL, with mobile phase A consisting of 95% water+20 mM ammonium acetate (pH 9.4), and mobile phase B being 98% acetonitrile. The flow rate was set at 0.15 mL/min with a gradient program running as follows: 0 minutes, 90% B; 2 minutes, 90% B; 3 minutes, 75% B; 7 minutes, 75% B; 8 minutes, 70% B; 9 minutes, 70% B; 10 minutes, 50% B; 12 minutes, 50% B; 13 minutes, 25% B; 14 minutes, 25% B; 16 minutes, 0% B; 20 minutes, 0% B; 21 minutes, 90% B; and 25 minutes, 90% B, with a 2-minute post-run period. The second method, HILIC-02, used an Atlantis Premier BEH Z-HILIC column (1.7 μm, 2.1×100 mm) at a column temperature of 30° C. and an autosampler temperature of 8° C. The injection volume was again 5 μL, with mobile phase A consisting of 70% water+5 mM ammonium formate (pH 4.0) and mobile phase B composed of 95% acetonitrile+5 mM ammonium formate (pH 4.0). The flow rate was set to 0.25-0.4 mL/min, with the gradient program as follows: 0 minutes, 100% B (flow rate 0.25 mL/min); 1 minute, 100% B (flow rate 0.25 mL/min); 10.5 minutes, 60% B (flow rate 0.25 mL/min); 13 minutes, 15% B (flow rate 0.25 mL/min); 13.5 minutes, 15% B (flow rate 0.25 mL/min); 14 minutes, 100% B (flow rate 0.4 mL/min); 18.5 minutes, 100% B (flow rate 0.4 mL/min); 19 minutes, 100% B (flow rate 0.25 mL/min); 20 minutes, 100% B (flow rate 0.25 mL/min).

Targeted detection and quantification for plasma metabolites and lipids analysis was performed on an Agilent 6495 QqQ Mass Spectrometer using dynamic multiple reaction monitoring (dMRM) with an Agilent Jet Stream ion source. The polarity was switched between positive and negative modes. The gas temperature was set to 200° C., with a drying gas flow rate of 14 L/min, nebulizer gas at 50 psi, and sheath gas at 375° C. and 12 L/min. The capillary voltage was set to 3,000 V for positive and −2,500 V for negative polarity, with a nozzle voltage of 0 V for both polarities. The iFunnel high/low pressure RF was set to 150/60 V for positive and 90/60 V for negative polarity. The scan type was set to dMRM with unit resolution for both Q1 and Q2, a delta EMV of 0 V (positive) and 200 V (negative), and a cell acceleration voltage of 5 V. The dMRM method was generated using Agilent MassHunter Acquisition software.

### 5. Development of Plasma Proteomics Fingerprint Database: CompleteBank-Discovered

For Plasma Proteins: over 9,000 plasma proteomic runs using the timsTOF HT mass spectrometer (Billerica, Massachusetts, USA) were analyzed, systematically documenting the performance of each detected plasma protein. A stringent false discovery rate (FDR) threshold of 1% was applied in discovery-mode analysis to ensure high confidence in protein identification. By integrating data from these runs, 17,328 unique plasma proteins were identified. To facilitate validation on triple quadrupole (QqQ) mass spectrometers, Applicants categorized these proteins into distinct classes based on their physicochemical properties and detection characteristics. A robust algorithm was developed to effectively translate protein fingerprints identified on the time-of-flight (TOF) platform to QqQ mass spectrometers. Validation assays were subsequently conducted using QqQ platforms from multiple vendors to confirm the reproducibility and reliability of these identified proteins.

Plasma Metabolites and Lipids: To establish a comprehensive plasma metabolite and lipid profile, Applicants compiled a curated list of nearly 3,000 small molecules, including metabolites and lipids, based on an extensive literature review. Each molecule was subjected to at least six different analytical approaches to determine the optimal detection conditions in human plasma samples. To maximize the number of detectable small molecules while minimizing assay run time and improving throughput, Applicants manually curated and optimized the resulting data. This refinement process led to the development of a high-efficiency detection protocol, ultimately documenting 2,927 molecules in the CompleteBank-Discovery database.

### 6. Establishment of Validated and Optimized Detection Assay Clusters: CompleteBank-Validated

For Plasma Proteins: To transition from discovery to validated clinical applications, Applicants evaluated the clinical relevance of each identified plasma protein and selected an initial panel of 12,892 proteins from the discovery cohort. Extensive QqQ-based method optimization was performed for these proteins, involving iterative assays to fine-tune detection conditions, enhance sensitivity, and ensure reproducibility. Through this rigorous process, Applicants refined the panel to a final set of 10,598 validated proteins, optimized for reliable quantification using QqQ mass spectrometry.

Plasma Metabolites and Lipids: For targeted metabolomics and lipidomics, Applicants established validated detection parameters for 762 metabolites and 1,395 lipids. To achieve optimal characterization, Applicants employed three different chromatographic columns and implemented six distinct analytical methods, ensuring comprehensive coverage and precise quantification of these molecules.

### Example 2: CompletePeaking: An Automatic Bioinformatic Pipeline for Clinical Proteomics and Metabolomics Data Processing

For Plasma Proteins: One of the essential parts of the Complete360® pipeline is its methodology for peak picking and data analysis pipeline, and Applicants term this entire bioinformatic pipeline as CompletePeaking. The CompletePeaking process combines manual curation with automated machine learning approaches to ensure the accurate identification and quantification of peptides in complex proteomic datasets. Initially, Applicants employed the Complete360® methods for manual validation of proteomics data, where human evaluators examined chromatograms and spectra for peptide transitions. The goal was to ensure that the peaks of interest coeluted with reference transitions and exhibited minimal background noise. Manual validation focused on assessing coelution of peaks and verifying that they exhibited high library dot product scores, which quantify the similarity between observed and reference intensity profiles.

Each peptide transition was scrutinized for quality indicators, including signal-to-noise ratio (SNR), intensity, and the presence of coelution, with a tolerance threshold of 0.3 minutes for retention time, as well as surrounding noise signals for each specific transitions. The manual curation process, although labor-intensive, was crucial in establishing the initial training dataset across diverse clinical samples, including several advanced stage cancer plasma samples, cardiovascular disease samples, neurodegenerative disease plasma samples and also inflammation and auto-immune disease plasma samples. These curated datasets formed the foundation for subsequent CompletePeaking model training.

Validation Process: During the manual validation phase, evaluators closely monitored the retention times of peptide transitions to ensure they coeluted. The measured retention times were then compared to predicted peak retention times, ensuring they fell within a defined tolerance. Dot product calculations were performed to assess the similarity between the observed and reference spectra, with high dot product scores indicating a strong match to the expected peptide identity. Peaks exhibiting both strong coelution and high dot product scores were labeled as “good” peaks, which were subsequently used as input for model training.

Despite the advantages of manual validation, it introduces certain biases. Evaluators often tend to prioritize peaks with higher intensity, which, while visually striking, may not always correspond to biologically relevant transitions. To mitigate these biases, Applicants incorporated several strategies to improve the reliability of the training data. These included matching retention times across multiple samples, identifying reproducible noise landscapes around target peaks, and closely examining the patterns of transition similarity to the reference library. This comprehensive approach helped to ensure more accurate and consistent peak selection, enhancing the reliability of the training data.

A critical aspect of this study is the long-term accumulation of data, with years of manual peak validation forming the core of the current training dataset. By manually curating and validating a large number of peaks across diverse clinical samples, Applicants developed a robust dataset that captured the nuances of peak coelution, retention times, and library dot product scores. This curated dataset served as the backbone for training machine learning models, enabling the automation of peak selection while preserving the high accuracy and reliability achieved through manual methods.

Automated Peak Picking Using Machine Learning: To address the limitations of manual peak selection and improve scalability, Applicants integrated an artificial intelligence-based machine learning model to automate the peak picking process. The model was trained using the XGBoost algorithm with a dataset that had been manually validated, incorporating various features derived from chromatogram analysis, such as coelution count, signal-to-noise ratio (SNR), shape correlation, and intensity. These features were essential for distinguishing high-quality peaks from those impacted by noise or background interference. A particularly important feature was coelution count, which represents the number of transitions that coelute at consistent retention times. A higher coelution count significantly boosts the confidence in identifying a valid peak. Additional features, including SNR, dot product, and shape correlation, were also considered, with their weights adjusted according to their contribution to the model's overall performance. The model was trained using cross-validation to optimize hyperparameters and prevent overfitting, ensuring that it remained robust across different datasets. This process allowed the model to generalize well, improving its ability to reliably identify peaks across a variety of samples and conditions.

This combination of manual curation and automated machine learning not only enhances the accuracy and scalability of the peak picking process but also overcomes the limitations of traditional proteomics workflows. By integrating manual validation with machine learning models trained on years of curated data, Applicants provide a more consistent, reliable, and high-throughput solution for proteomics studies. This ensures the identification of high-quality peaks across a variety of clinical samples, facilitating the analysis of large, complex datasets.

Data Preprocessing and Feature Generation: The preprocessing pipeline utilized CompleteBank-Discovered results to generate feature files, which provided a comprehensive list of candidate peaks based on observed chromatogram data. These candidate peaks were subsequently labeled according to manual validation results, with “good” peaks identified as those that met the quality criteria of coelution and high dot product scores. Features extracted from these peaks-including coelution count, dot product, signal-to-noise ratio (SNR), and shape correlation-were used as input for the XGBoost model. The model was trained to perform binary classification, distinguishing peaks as either valid or invalid based on their predicted retention times and associated feature characteristics. The model's performance in predicting accurate peak retention times and selecting the most reliable candidate peaks was evaluated using a dataset comprising over 15,000 peptides, manually validated across a variety of representative clinical samples, including pooled advanced disease plasma samples. This robust dataset ensured that the model was trained on diverse, real-world data, enhancing its accuracy and generalizability.

Peak Selection, Postprocessing, and Data Normalization: After model training, automated peak selection was applied to new, unseen data. The postprocessing steps included selecting the highest-scoring peaks for each peptide sequence, ensuring that the final selection consisted of the most reliable peaks with the highest likelihood of accurate identification. Following peak selection, data normalization was performed on the peak area data collected from each target analyte. Given the variety of detection methods and clustering based on peak intensities, hydrophobicities, retention times, and other factors, normalization was conducted for each cluster using separate methods. As a result, multiple reference points were used for normalization, tailored to each cluster of target analysis. This approach led to the development of the Multi-point Normalized Protein expression (MNPX) value, which represents the normalized expression for each protein. After normalization, the abundance of each protein across various samples could be compared, enabling the evaluation of its correlation with disease states and facilitating the identification of potential protein biomarkers. For the current study, the normalization factor for each protein was chosen as the median intensity of the biomarker's intensity in that detection cluster. Further normalization can be updated to use stably expressed plasma proteins or disease specific normalizers but will subject to further evaluation and development with at least several hundred samples for each disease type and will be updated in a further study.

For Plasma Metabolites and Lipids: In CompletePeaking pipeline, Applicants also developed a set of metabolomics peak picking algorithms based on a second derivative method. This approach was used to identify peaks in metabolomics datasets by analyzing the intensity profiles that are changing over time.

Smoothing the Intensity Signal: The raw intensity data was smoothed using the Savitzky-Golay filter to reduce noise while preserving peak shape. The smoothing was applied with a window length of 11 and a polynomial order of 3.

Computing the Second Derivative: The second derivative of the smoothed intensity was computed to capture the changes in concavity that mark the boundaries of a peak. These inflection points were used to define the start and end points of each peak.

Zero-Crossings for Boundary Detection: Zero-crossings of the second derivative were identified, marking the transition from concave up to concave down. These zero-crossings defined the start and end time points of each peak. Adjustments were made to the boundaries to account for peak asymmetry, with an extension factor applied to both the start and end points.

Background Correction and Tail Adjustment: A background intensity threshold was calculated as the median intensity outside the peak region. The peak boundaries were iteratively adjusted to ensure that they did not extend into noise regions, and the tail symmetry condition was met to balance the peak shape.

Width Correction: To ensure consistent peak-width estimation across samples, outliers in peak width were removed using the interquartile range (IQR) method. After removing outliers, the peak widths were standardized based on the sample with the largest total area.

This second derivative method provided a robust framework for metabolomics peak detection, and it was incorporated as an essential part of CompletePeaking pipeline to identify and quantify metabolomic signals.

Detectability in Complete360® Methods: The Complete360® platform employs a rigorous set of criteria to ensure high-confidence detection and quantification of target proteins. These criteria were established to maximize sensitivity, reproducibility, and specificity in deep proteomic profiling. The following parameters define detectability within the Complete360® workflow:

Signal-to-Noise Ratio (S/N): Reliable detection of analytes requires a minimum signal-to-noise (S/N) ratio of 1.5, ensuring adequate signal intensity above background fluctuations.

Retention Time (RT) Consistency: Retention time (RT) for analyte detection must exceed 1.5 minutes, preventing interference from early eluting compounds and maintaining consistency across runs.

Background and Interference Control: To ensure specificity, analyte peaks must exhibit minimal interference from co-eluting species. Background signals within the retention window are required to be at least one order of magnitude lower than the analyte peak intensity. For example, an analyte peak with an intensity of 1,000 counts must have a background signal of ≤100 counts.

Co-Elution of Fragment Ions: The apexes of monitored transitions must closely align within a time window of ±0.05-0.2 minutes, ensuring the consistency of elution profiles and preventing misidentification.

Fragment Ion Ratio Consistency: Ion ratios between transitions of the same analyte must remain within ±20-30% of reference values, ensuring stability in detection across different analytical runs.

Spectral Similarity Assessment: To validate fragmentation patterns, spectral comparisons with high-resolution discovery data are performed. A dot-product or similarity score of ≥0.5-0.6 is required to confirm alignment with expected fragmentation profiles.

Separation of high- and low-abundance molecules: High- and low-abundance molecules should be detected separately to minimize ion suppression effects on low-abundance targets. This ensures that low-abundance molecules are detected with enhanced sensitivity and reproducibility, avoiding interference from high-abundance molecules, which typically overshadow other target analytes²³.

Resolution of co-eluted targets: Co-eluted targets should be resolved at higher resolution when their abundance permits. While higher resolution may reduce the number of ions entering the mass analyzer of a mass spectrometer, potentially affecting sensitivity, careful manual tuning and optimization for each protein can address this.

Retention time reproducibility: Retention times for various analytes must be consistently reproducible across different assay environments. For instance, an analyte detected in a low pH fraction may exhibit a significantly different retention time compared to its detection in a high pH fraction. This variation underscores the importance of considering the surrounding microenvironment to ensure reproducible detection schedule.

Management of co-eluting noise analytes: High-abundance noise analytes must be carefully controlled to avoid co-elution with low-abundance target analytes. In current application, some high-abundance analytes have been utilized to serve as highly reproducible landmarks, which can enhance data annotation accuracy and improve overall analytical precision which is further illustrated later.

Minimizing Run Time: To integrate all target analyses into a single assay with minimal runtime, an AI-driven pattern-clustering approach was employed. This method reduced overall run time while optimizing the sensitivity and reproducibility of each analyte when consolidated into the same assay. This AI-driven algorithm is an essential component of Applicants' in-house developed software package, which can also be used to establish customized diagnostic methods, such as those focused on specific diseases.

These criteria collectively ensure the robustness and accuracy of the Complete360® methodology, facilitating the precise detection of ultra-low abundance proteins in complex biological matrices.

### REFERENCES

1. Nicholson, B. D. et al. Multi-cancer early detection test in symptomatic patients referred for cancer investigation in England and Wales (SYMPLIFY): a large-scale, observational cohort study. Lancet Oncol 24, 733-743 (2023).
2. Klein, E. A. et al. Clinical validation of a targeted methylation-based multi-cancer early detection test using an independent validation set. Ann Oncol 32, 1167-1177 (2021).
3. Cohen, J. D. et al. Detection and localization of surgically resectable cancers with a multi-analyte blood test. Science 359, 926-930 (2018).
4. Chen, M. & Zhao, H. Next-generation sequencing in liquid biopsy: cancer screening and early detection. Hum Genomics 13, 34 (2019).
5. Davies, M. P. A. et al. Plasma protein biomarkers for early prediction of lung cancer. EBioMedicine 93, 104686 (2023).
6. Wang, Q. et al. Selected reaction monitoring approach for validating peptide biomarkers. Proc Natl Acad Sci USA 114, 13519-13524 (2017).
7. FDA (2024).
8. Vitko, D. et al. timsTOF HT Improves Protein Identification and Quantitative Reproducibility for Deep Unbiased Plasma Protein Biomarker Discovery. J Proteome Res 23, 929-938 (2024).
9. Heil, L. R. et al. Evaluating the Performance of the Astral Mass Analyzer for Quantitative Proteomics Using Data-Independent Acquisition. J Proteome Res 22, 3290-3300 (2023).
10. Bonaventura, P. et al. Identification of shared tumor epitopes from endogenous retroviruses inducing high-avidity cytotoxic T cells for cancer immunotherapy. Sci Adv 8, eabj3671 (2022).
11. Hsiuc, E. H. et al. Targeting a neoantigen derived from a common TP53 mutation. Science 371 (2021).
12. Terai, Y. L. et al. Valid-NEO: A Multi-Omics Platform for Neoantigen Detection and Quantification from Limited Clinical Samples. Cancers (Basel) 14 (2022).
13. Wang, Q. et al. Direct Detection and Quantification of Neoantigens. Cancer Immunol Res 7, 1748-1754 (2019).
14. Omenn, G. S. et al. The 2024 Report on the Human Protcome from the HUPO Human Protcome Project. J Proteome Res 23, 5296-5311 (2024).
15. Wishart, D. S. et al. MarkerDB: an online database of molecular biomarkers. Nucleic Acids Res 49, D1259-D1267 (2021).
16. Schooneveldt, Y. L. et al. The Impact of Simvastatin on Lipidomic Markers of Cardiovascular Risk in Human Liver Cells Is Secondary to the Modulation of Intracellular Cholesterol. Metabolites 11 (2021).
17. Su, B. et al. A DMS Shotgun Lipidomics Workflow Application to Facilitate High-Throughput, Comprehensive Lipidomics. J Am Soc Mass Spectrom 32, 2655-2663 (2021).
18. Cao, Z. et al. Evaluation of the Performance of Lipidyzer Platform and Its Application in the Lipidomics Analysis in Mouse Heart and Liver. J Proteome Res 19, 2742-2749 (2020).
19. Medina, J. et al. Omic-Scale High-Throughput Quantitative LC-MS/MS Approach for Circulatory Lipid Phenotyping in Clinical Research. Anal Chem 95, 3168-3179 (2023). Tianqi Chen, C. G. XGBoost: A Scalable Trec Boosting System. arXiv:1603.02754 20. (2016).
21. Ignjatovic, V. et al. Mass Spectrometry-Based Plasma Protcomics: Considerations from Sample Collection to Achieving Translational Data. J Proteome Res 18, 4085-4097 (2019).
22. Millioni, R. et al. High abundance proteins depletion vs low abundance proteins enrichment: comparison of methods to reduce the plasma protcome complexity. PLOS One 6, c19603 (2011).
23. Tu, C. et al. Depletion of abundant plasma proteins and limitations of plasma proteomics. J Proteome Res 9, 4982-4991 (2010).
24. Douglass, J. et al. Bispecific antibodies targeting mutant RAS neoantigens. Sci Immunol 6 (2021).
25. Deutsch, E. W. et al. Advances and Utility of the Human Plasma Proteome. J Proteome Res 20, 5241-5263 (2021).
26. Pettersson, F. et al. Ribavirin treatment effects on breast cancers overexpressing eIF4E, a biomarker with prognostic specificity for luminal B-type breast cancer. Clin Cancer Res 17, 2874-2884 (2011).
27. Gao, Y. et al. Loss of ERalpha induces amoeboid-like migration of breast cancer cells by downregulating vinculin. Nat Commun 8, 14483 (2017).
28. Al-Khatib, S. M. et al. Exploring Genetic Determinants: A Comprehensive Analysis of Serpin B Family SNPs and Prognosis in Glioblastoma Multiforme Patients. Cancers (Basel) 16 (2024).
29. Ding, Q. et al. Ficolin-2 triggers antitumor effect by activating macrophages and CD8(+) T cells. Clin Immunol 183, 145-157 (2017).
30. Cao, B. et al. Cancer-mutated ribosome protein L22 (RPL22/eL22) suppresses cancer cell survival by blocking p53-MDM2 circuit. Oncotarget 8, 90651-90661 (2017).
31. Zheng, X. et al. Serine/arginine-rich splicing factors: the bridge linking alternative splicing and cancer. Int J Biol Sci 16, 2442-2453 (2020).
32. Moon, C. M., Yun, K. E., Ryu, S., Chang, Y. & Park, D. I. High serum alanine aminotransferase is associated with the risk of colorectal adenoma in Korean men. J Gastroenterol Hepatol 32, 1310-1317 (2017).
33. Xu, Z. et al. CCL19 suppresses angiogenesis through promoting miR-206 and inhibiting Met/ERK/Elk-1/HIF-1alpha/VEGF-A pathway in colorectal cancer. Cell Death Dis 9, 974 (2018).
34. Bui, Q. T., Hong, J. H., Kwak, M., Lec, J. Y. & Lec, P. C. Ubiquitin-Conjugating Enzymes in Cancer. Cells 10 (2021).
35. Zhou, J. et al. RhoE is associated with relapse and prognosis of patients with colorectal cancer. Ann Surg Oncol 20, 175-182 (2013).
36. Ubink, I., Verhaar, E. R., Kranenburg, O. & Goldschmeding, R. A potential role for CCN2/CTGF in aggressive colorectal cancer. J Cell Commun Signal 10, 223-227 (2016).
37. Kumarakulasingham, M. et al. Cytochrome p450 profile of colorectal cancer: identification of markers of prognosis. Clin Cancer Res 11, 3758-3765 (2005).
38. Zhan, Q., Ma, X. & He, Z. PEAR1 suppresses the proliferation of pulmonary microvascular endothelial cells via PI3K/AKT pathway in ALI model. Microvasc Res 128, 103941 (2020).
39. Cedzynski, M. & Swierzko, A. S. Components of the Lectin Pathway of Complement in Solid Tumour Cancers. Cancers (Basel) 14 (2022).
40. Sabater, L., Gomez-Choco, M., Saiz, A. & Graus, F. BR serine/threonine kinase 2: a new autoantigen in parancoplastic limbic encephalitis. J Neuroimmunol 170, 186-190 (2005).
41. Chen, M. et al. Case Report: THSD7A-Positive Membranous Nephropathy Caused by Tislelizumab in a Lung Cancer Patient. Front Immunol 12, 619147 (2021).
42. Van, A. N. et al. Protein kinase C fusion proteins are paradoxically loss of function in cancer. J Biol Chem 296, 100445 (2021).
43. Kim, N. et al. Integrated genomic approaches identify upregulation of SCRN1 as a novel mechanism associated with acquired resistance to erlotinib in PC9 cells harboring oncogenic EGFR mutation. Oncotarget 7, 13797-13809 (2016).
44. Horiuchi, A. et al. Up-regulation of small GTPases, RhoA and RhoC, is associated with tumor progression in ovarian carcinoma. Lab Invest 83, 861-870 (2003).
45. Gomes, F. C. et al. Social, Genetics and Histopathological Factors Related to Titin (TTN) Gene Mutation and Survival in Women with Ovarian Serous Cystadenocarcinoma: Bioinformatics Analysis. Genes (Basel) 14 (2023).
46. Xic, Y. et al. Structural basis for high-order complex of SARNP and DDX39B to facilitate mRNP assembly. Cell Rep 42, 112988 (2023).
47. Jin, C. et al. UCHL1 Is a Putative Tumor Suppressor in Ovarian Cancer Cells and Contributes to Cisplatin Resistance. J Cancer 4, 662-670 (2013).
48. Gozzi, G. et al. Promoter methylation and downregulated expression of the TBX15 gene in ovarian carcinoma. Oncol Lett 12, 2811-2819 (2016).
49. Dominguez, C. L. et al. Diacylglycerol kinase alpha is a critical signaling node and novel therapeutic target in glioblastoma and other cancers. Cancer Discov 3, 782-797 (2013).
50. Chen, Y. et al. N(6)-methyladenosine-modified TRAF1 promotes sunitinib resistance by regulating apoptosis and angiogenesis in a METTL14-dependent manner in renal cell carcinoma. Mol Cancer 21, 111 (2022).
51. Sung, H. Y., Han, J., Ju, W. & Ahn, J. H. Synaptotagmin-like protein 2 gene promotes the metastatic potential in ovarian cancer. Oncol Rep 36, 535-541 (2016).
52. Chang, J. et al. Exome-wide analysis identifies three low-frequency missense variants associated with pancreatic cancer risk in Chinese populations. Nat Commun 9, 3688 (2018).
53. Zeng, G. et al. Aberrant Wnt/beta-catenin signaling in pancreatic adenocarcinoma. Neoplasia 8, 279-289 (2006).
54. Wang, W. et al. USP35 mitigates endoplasmic reticulum stress-induced apoptosis by stabilizing RRBPI in non-small cell lung cancer. Mol Oncol 16, 1572-1590 (2022).
55. Zhang, R. et al. KIF22 Promotes Development of Pancreatic Cancer by Regulating the MEK/ERK/P21 Signaling Axis. Biomed Res Int 2022, U.S. Pat. No. 6,000,925 (2022).
56. Javanshir, H. T. et al. Investigation of key signaling pathways and appropriate diagnostic biomarkers selection between non-invasive to invasive stages in pancreatic cancer: a computational observation. J Med Life 15, 1143-1157 (2022).
57. Tokizane, T. et al. Cytochrome P450 1B1 is overexpressed and regulated by hypomethylation in prostate cancer. Clin Cancer Res 11, 5793-5801 (2005).
58. Gross, M. et al. Beta-2-microglobulin is an androgen-regulated secreted protein elevated in serum of patients with advanced prostate cancer. Clin Cancer Res 13, 1979-1986 (2007).
59. Kim, J. W. et al. Genetic and epigenetic inactivation of LPL gene in human prostate cancer. Int J Cancer 124, 734-738 (2009).
60. Chen, V. & Shtivelman, E. CC3/TIP30 regulates metabolic adaptation of tumor cells to glucose limitation. Cell Cycle 9, 4941-4953 (2010).
61. Marin, L. & Casado, F. Prediction of prostate cancer biochemical recurrence by using discretization supports the critical contribution of the extra-cellular matrix genes. Sci Rep 13, 10144 (2023).
62. Linxweiler, M., Schick, B. & Zimmermann, R. Let's talk about Secs: Sec61, Sec62 and Sec63 in signal transduction, oncology and personalized medicine. Signal Transduct Target Ther 2, 17002 (2017).
63. Okada, K., Itoh, H. & Ikemoto, M. Scrum complement C3 and alpha (2)-macroglobulin are potentially useful biomarkers for inflammatory bowel disease patients. Heliyon 7, e06554 (2021).
64. Elkasrawy, M. N. & Hamrick, M. W. Myostatin (GDF-8) as a key factor linking muscle mass and bone structure. J Musculoskelet Neuronal Interact 10, 56-63 (2010).
65. Schnetkamp, P. P. The SLC24 Na+/Ca2+-K+ exchanger family: vision and beyond. Pflugers Arch 447, 683-688 (2004).
66. Ma, X. N., Li, M. Y., Qi, G. Q., Wei, L. N. & Zhang, D. K. SUMOylation at the crossroads of gut health: insights into physiology and pathology. Cell Commun Signal 22, 404 (2024).
67. Tsuchihara, K. et al. Ckap2 regulates aneuploidy, cell cycling, and cell death in a p53-dependent manner. Cancer Res 65, 6685-6691 (2005).
68. Wang, W., Zhao, F., Ma, X., Perry, G. & Zhu, X. Mitochondria dysfunction in the pathogenesis of Alzheimer's disease: recent advances. Mol Neurodegener 15, 30 (2020).
69. Sekine, M. & Makino, T. Inference of Causative Genes for Alzheimer's Disease Due to Dosage Imbalance. Mol Biol Evol 34, 2396-2407 (2017).
70. Zattoni, M. et al. Serpin Signatures in Prion and Alzheimer's Diseases. Mol Neurobiol 59, 3778-3799 (2022).
71. Szymura, S. J. et al. DDX39B interacts with the pattern recognition receptor pathway to inhibit NF-kappaB and sensitize to alkylating chemotherapy. BMC Biol 18, 32 (2020).
72. Ferragut, F., Vachetta, V. S., Troncoso, M. F., Rabinovich, G. A. & Elola, M. T. 72. ALCAM/CD166: A pleiotropic mediator of cell adhesion, stemness and cancer progression. Cytokine Growth Factor Rev 61, 27-37 (2021).
73. Pouw, R. B. et al. Complement Factor H-Related Protein 4A Is the Dominant Circulating Splice Variant of CFHR4. Front Immunol 9, 729 (2018).
74. Ganley, I. G., Espinosa, E. & Pfeffer, S. R. A syntaxin 10-SNARE complex distinguishes two distinct transport routes from endosomes to the trans-Golgi in human cells. J Cell Biol 180, 159-172 (2008).
75. Niu, L. et al. Plasma proteome variation and its genetic determinants in children and adolescents. Nat Genet (2025).
76. Obradovic, M. et al. Leptin and Obesity: Role and Clinical Implication. Front Endocrinol (Lausanne) 12, 585887 (2021).
77. Candia, J. et al. Assessment of Variability in the SOMAscan Assay. Sci Rep 7, 14248 (2017).
78. Candia, J., Daya, G. N., Tanaka, T., Ferrucci, L. & Walker, K. A. Assessment of variability in the plasma 7 k SomaScan proteomics assay. Sci Rep 12, 17147 (2022).
79. Smits, H. M. et al. The BAMBOO method for correcting batch effects in high throughput proximity extension assays for protcomic studies. Sci Rep 15, 1498 (2025).
80. Dennis, M. S. et al. Albumin binding as a general strategy for improving the pharmacokinetics of proteins. J Biol Chem 277, 35035-35043 (2002).
81. Hui, H., Farilla, L., Merkel, P. & Perfetti, R. The short half-life of glucagon-like peptide-1 in plasma does not reflect its long-lasting beneficial effects. Eur J Endocrinol 146, 863-869 (2002).
82. Razavi, M. et al. Measuring the Turnover Rate of Clinically Important Plasma Proteins using an Automated SISCAPA Workflow. Clin Chem 65, 492-494 (2019).
83. Wang, Y. et al. Reversed-phase chromatography with multiple fraction concatenation strategy for protcome profiling of human MCF10A cells. Proteomics 11, 2019-2026 (2011).
84. Demichev, V., Messner, C. B., Vernardis, S. I., Lilley, K. S. & Ralser, M. DIA-NN: neural networks and interference correction enable deep proteome coverage in high throughput. Nat Methods 17, 41-44 (2020).
85. Kong. A. T., Leprevost. F. V., Avtonomov. D. M., Mellacheruvu, D. & Nesvizhskii, A. I. MSFragger: ultrafast and comprehensive peptide identification in mass spectrometry-based proteomics. Nat Methods 14, 513-520 (2017).

1. A method for multi-omics biomarker detection in a single analytical pipeline, the method comprising:
(a) obtaining a biological sample comprising proteins, metabolites, and lipids,
wherein the sample is preserved for analyte detection across a concentration range from 1 ng/L to 100 mg/L,
(b) subjecting said sample to a unified preparation step that simultaneously removes high-abundance components and maintains the stability of proteins, metabolites, and lipids,
wherein no separate instrumentation or reconfiguration is performed for individual biomolecular classes,
(c) performing a mass spectrometry-based detection of said proteins, metabolites, and lipids in a single run or in multiple consecutive runs on the same instrumentation without major hardware reconfiguration,
wherein a machine-learning model, trained on at least hundreds of thousands manually curated mass spectrometry datasets, automatically discriminates true analyte signals from noise, achieving a coefficient of variation of ten percent or less for repeated measurements,
(d) comparing the resulting proteomic, metabolomic, and lipidomic data to an iterative biomarker database that transitions biomarkers from a discovery stage to a validated stage upon meeting sensitivity and reproducibility thresholds, and
(e) generating a disease-specific classification or biomarker panel from the integrated multi-omics signals.

2. The method of claim 1, wherein processing the sample in step (b) comprises a two-step depletion protocol including chemical precipitation and antibody-conjugated resin depletion of high-abundance plasma proteins.

3. The method of claim 1, wherein the biological sample in step (a) is a dried blood spot, and said processing includes incubating said dried blood spot in a stabilization reagent at ambient temperature for at least three days without substantial biomarker degradation.

4. The method of claim 1, wherein the mass spectrometry-based workflow in step (c) is operable across a dynamic range spanning about 1 ng/L to about 100 mg/L, enabling detection of ultra-low and high-abundance molecules in a single run.

5. The method of claim 1, wherein applying the data analysis pipeline in step (d) includes training the machine-learning model on at least hundreds of thousands manually curated spectra, reducing the coefficient of variation below about 10%.

6. The method of claim 1, further comprising iteratively refining detection parameters by repeating steps (b) through (d) and updating the biomarker database upon meeting predefined reproducibility criteria.

7. The method of claim 1, wherein identifying a disease-specific biomarker panel in step (e) includes generating a receiver operating characteristic (ROC) curve with improved area under the curve (AUC) when integrating proteomic, metabolomic, and lipidomic features.

8. The method of claim 1, further comprising correlating one or more identified protein biomarkers with genetic variants via proteomic quantitative trait loci (pQTL) analysis, refining disease risk predictions.

9. The method of claim 1, wherein processing the sample in step (b) further includes doping the sample with internal standard peptides for quantitative calibration of target analytes.

10. The method of claim 1, wherein the mass spectrometry-based workflow in step (c) employs dynamic multiple reaction monitoring (dMRM) that automatically adjusts collision energies in real time to enhance detection of low-abundance biomarkers.

11. A system for integrated multi-omics biomarker detection, comprising:
(a) a unified sample preparation module configured to remove high-abundance components and preserve proteins, metabolites, and lipids from a single biological sample,
wherein no separate instrumentation or reconfiguration is required for individual biomolecular classes;
(b) a mass spectrometer assembly operable to detect proteins, metabolites, and lipids in one run or in multiple consecutive runs on the same instrumentation without major hardware reconfiguration across a concentration range from 1 ng/L to 100 mg/L,
wherein said assembly detects said analytes without necessitating distinct hardware setups for proteomic versus small-molecule analysis;
(c) a multi-phase biomarker database stored on at least one memory device, the database comprising discovery-phase entries and validated-phase entries; and
(d) a computing unit communicatively coupled to the mass spectrometer assembly and the biomarker database,
wherein the computing unit is programmed to:
(i) execute a machine-learning model trained on at least hundreds of thousands curated mass spectrometry datasets to distinguish analyte signals from noise with a quantification accuracy of coefficient of variation of ten percent or less,
(ii) update said biomarker database by transitioning discovered biomarkers to validated-phase entries upon meeting predefined reproducibility thresholds, and
(iii) generate a disease-specific classification or biomarker panel such that an area-under-the-curve (AUC) of at least 0.7 is achieved when distinguishing diseased samples from non-diseased samples.

12. The system of claim 11, wherein the sample preparation module comprises a chemical precipitation unit followed by an antibody-conjugated resin for selectively removing high-abundance plasma proteins.

13. The system of claim 11, further comprising a dried blood spot interface, wherein said sample preparation module includes a stabilization reagent adapted to minimize protein degradation for at least five days at ambient temperature.

14. The system of claim 11, wherein the mass spectrometer assembly is configured to detect biomolecules over a dynamic range from about 1 ng/L to about 100 mg/L, enabling quantification of ultra-low abundance proteins.

15. The system of claim 11, wherein the computing unit is programmed to execute a peak analysis model trained on over 1,000,000 mass spectrometry runs, achieving a reproducibility coefficient of variation below about 10%.

16. The system of claim 11, wherein the biomarker database is iteratively updated based on repeated sample analyses, transitioning candidate biomarkers from a discovery phase to a validated phase upon meeting reproducibility thresholds.

17. The system of claim 11, wherein the computing unit classifies disease states by selecting a subset of proteins, metabolites, and lipids that maximize diagnostic performance in a receiver operating characteristic (ROC) analysis, exceeding a preselected area under the curve (AUC) threshold.

18. The system of claim 11, further comprising a pQTL analysis module integrated within the computing unit, configured to correlate identified protein biomarkers with genomic variants.

19. The system of claim 11, wherein the mass spectrometer assembly is automatically tuned to adjust ionization parameters in real time through dynamic multiple reaction monitoring (dMRM), improving detection of low-abundance targets.

20. The system of claim 11, wherein the computing unit applies internal standard peptides to ensure both relative and absolute quantification of proteins, metabolites, and lipids, enabling cross-run comparisons in a multi-omics dataset.
